SEQUENCE LISTING (2 sequences)

SEQ ID NO: 1 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 2 - protein, 517 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

(19) United States
(12) Patent Application Publication
(10) Pub. No.: US 2026/0014242 A1
(43) Pub. Date: Jan. 15, 2026

(54) RSV VACCINATION WITH TRIMERIC RSV F FUSION PROTEIN

(71) Applicant: GLAXOSMITHKLINE BIOLOGICALS SA, Rixensart (BE)

(72) Inventors: Marie-Pierre Paule DAVID, Wavre (BE); Laurence Anne Michèle FISSETTE, Wavre (BE); Narcisa MESAROS, Wavre (BE); Aurélie Chantal Marceline Louise Julienne OLIVIER, Wavre (BE); Marie VAN DER WIELEN, Wavre (BE); Jean-Yves Marc Noël PIRÇON, Wavre (BE)

(73) Assignee: GLAXOSMITHKLINE BIOLOGICALS SA, Rixensart (BE)

(21) Appl. No.: 18/867,069

(22) PCT Filed: Jun. 8, 2023
(86) PCT No.: PCT/EP2023/065335

§ 371 Date: Nov. 19, 2024

Related U.S. Application Data
(60) Provisional application No. 63/351,314, filed on Jun. 10, 2022.

(51) Int. Cl. A61K 39/12; A61K 39/00; A61P 37/04

(52) U.S. Cl. CPC A61K 39/12; A61P 37/04; A61K 2039/55555; A61K 2039/55572; A61K 2039/55577

(57) ABSTRACT

The present invention relates to vaccination against respiratory syncytial virus (RSV), in particular to the use of a vaccine formulation comprising an RSV F fusion protein (RSV F protein) antigen and an adjuvant in methods of prevention of RSV infection and disease in older adults.

### CROSS-REFERENCE TO RELATED APPLICATIONS

This application is filed pursuant to 35 U.S.C. § 371 as a United States National Phase Application of International Application No. PCT/IB2023/065335 filed on Jun. 8, 2023, which claims the benefit of U.S. provisional application No. 63/351,314 filed on Jun. 10, 2022, the contents of which are hereby incorporated by reference.

### SEQUENCE LISTING

The instant application contains a Sequence Listing which has been submitted electronically in XML format and is hereby incorporated by reference in its entirety. Said XML copy, created on Nov. 12, 2024, is named 70178WO01_SL.xml and is 3,630 bytes in size.

### FIELD OF THE INVENTION

The present invention relates to vaccination against respiratory syncytial virus (RSV), in particular to the use of a vaccine formulation comprising an RSV F fusion protein (RSV F protein) antigen and an adjuvant in methods of prevention of RSV infection and disease in older adults.

### BACKGROUND TO THE INVENTION

Respiratory syncytial virus (RSV) is a highly contagious ribonucleic acid virus of the Pneumoviridae family that causes respiratory tract infections in people of all ages. In temperate climates throughout the world, RSV predictably causes fall-winter epidemics, whereas viral activity is more endemic in (sub-) tropical regions and outbreaks are less temporally focused. RSV exists in 2 antigenically distinct subgroups, referred to as RSV A and RSV B.

Because natural infection induces only partial protective immunity, re-infections occur frequently throughout a lifetime. Generally, these re-infections go undiagnosed because they usually manifest as common acute upper respiratory tract infections. However, in more vulnerable individuals (e.g. immunocompromised persons, persons with certain co-morbidities, or older adults (OA)), re-infections can also lead to severe disease. Although the role of RSV-specific T cells in preventing RSV-associated disease in OA remains unclear, some studies show that OA have decreased frequencies, functionality and proliferative capacity of RSV-specific T cell responses as compared to younger adults.

According to the US ‘Centers for Disease Control and Prevention’ (CDC), RSV leads to 177 000 hospitalizations and 14 000 deaths on average each year among adults ≥65 YOA in the United States. As the global population ages, the morbidity and mortality of respiratory infections appear to be steadily increasing. In the US, the burden of the disease has been shown to be significant and data indicate that RSV is comparable to influenza (in an influenza-vaccinated population) in terms of number of infections, hospitalization and deaths.

Similarly, a recent worldwide systematic review and meta-analysis to assess the global burden of RSV-associated acute respiratory infection (ARI) in adults aged ≥65 YOA found the estimated number of RSV-ARI cases to be 1.5 million with an incidence of 6.7 cases/1000 person-years in industrialized countries.

In spite of the significant medical need in OA aged 60 years or above, there is no prophylactic vaccine yet on the market for the prevention of ARI or lower respiratory tract disease (LRTD) caused by RSV. The antiviral drug ribavirin is currently the only approved antiviral therapy for RSV treatment, but its use is restricted to severe hospitalized cases in infants and young children. Two RSV-specific humanized monoclonal antibodies, palivizumab (Synagis) and motavizumab, are confirmed to be safe and effective in reducing RSV hospitalization rates and serious complications among high-risk children.

Currently available treatment for RSV in OA is generally supportive in nature, consisting of supplemental oxygen, intravenous fluids and bronchodilators. Inhaled and systemic corticosteroids are often prescribed in patients with asthma or chronic obstructive pulmonary disease (COPD). RSV has been associated with COPD exacerbation.

The RSV fusion protein (RSV F protein) is a Class I fusion glycoprotein abundant on the viral envelope and exists in a trimeric “prefusion” state on the viral surface. During infection, insertion of hydrophobic “fusion peptide” loops of RSV F into the cell membrane and extensive conformational change of the F protein results in refolding into an energetically favorable trimeric “postfusion” state, fusing the viral and cell membranes together in the process. The prefusion form of RSV F is the primary target of neutralizing antibodies in natural infection. For use in protein subunit vaccines, a soluble (not membrane-bound) antigen is desirable. Exogenously expressed soluble RSV F spontaneously folds into a highly stable trimer in the postfusion conformation. As such, RSV F antigens engineered to stably retain the prefusion conformation by mutation and/or replacement of the transmembrane and cytoplasmic domains by a heterologous trimerization domain have been explored for use in protein subunit vaccination. However, as yet, no RSV vaccine is available for use in any age group.

### SUMMARY OF THE INVENTION

The present invention provides a composition for use in a method of protection against, or prevention of, RSV disease in a human subject of 60 years of age or older. In particular, there is provided a composition comprising a trimeric RSV F protein in the prefusion conformation and an adjuvant comprising a saponin and a TLR-4 agonist for use in a method of protection against, or prevention of, RSV disease in a human subject, wherein the efficacy of the composition does not decline with age or wherein the efficacy increases with the age of the subject. As such, the compositions of the present invention may find use in a method of protection against, or prevention of, RSV disease in a human subject of 65 years of age or older, of 70 years of age or older, or of 75 years of age or older.

Thus, the invention also provides a method of protection against, or prevention of, RSV disease in a human subject of 60 years of age or older, comprising the step of administering to the subject a composition comprising a trimeric RSV F protein in the prefusion conformation and an adjuvant comprising a saponin and a TLR-4 agonist.

In one aspect, the present invention provides a method of protection against, or prevention of, RSV disease caused by RSV A and RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B. The method of providing protection against RSV disease caused by RSV A and RSV B in a human subject may comprise the step of administration of the composition to a subject of 60 years of age or older, for example of 65 years of age or older, 70 years of age or older, or of 75 years of age or older.

Thus, the present invention also provides a composition comprising a trimeric RSV F protein in the prefusion conformation and an adjuvant comprising a saponin and a TLR-4 agonist for use in a method of protection against, or prevention of, RSV disease caused by RSV A and RSV B in a human subject, wherein the trimeric RSV F protein in the prefusion conformation is derived from RSV A and the composition does not contain an RSV F protein derived from RSV B.

In another aspect, the present invention provides a composition comprising a trimeric RSV F protein in the prefusion conformation and an adjuvant comprising a saponin and a TLR-4 agonist, for use in a method of protection against, or prevention of, RSV disease in a human subject of 60 years of age or older having a pre-existing medical condition. Accordingly, the invention also provides a method of protection against, or prevention of, RSV disease in a human subject of 60 years of age or older having a pre-existing medical condition.

In a further aspect, the present invention provides a composition comprising a trimeric RSV F protein in the prefusion conformation and an adjuvant comprising a saponin and a TLR-4 agonist for use in a method of protection against, or prevention of, RSV disease in a human subject of 60 years of age or older, wherein the composition provides protection against, or prevention of, RSV acute respiratory infection (ARI), RSV lower respiratory tract disease (LRTD) or severe lower respiratory tract disease (severe LRTD). The invention also provides a method of protection against, or prevention of RSV acute respiratory infection (ARI), RSV lower respiratory tract disease (LRTD) or severe lower respiratory tract disease (severe LRTD) in a human subject of 60 years of age or older comprising the step of administering to the subject a composition comprising a trimeric RSV F protein in the prefusion conformation and an adjuvant comprising a saponin and a TLR-4 agonist.

### DESCRIPTION OF DRAWINGS/FIGURES

FIG. 1A The wildtype RSV F protein sequence is depicted in schematic form: signal peptide (25 residues), residue ectodomain including 3 heptad repeats (HRA, HRB, and HRC), transmembrane region (TM), short cytoplasmic tail, glycosylation sites (circled G), and furin cleavage sites (scissors). The signal peptide (SP) and p27 peptide are removed during protein maturation, resulting in the F1 and F2 chains (diamond symbol=post-cleavage site left after p27 removal).

FIG. 1B The recombinant RSV soluble F protein polypeptide is shown with the S155C, S290C, S190F, and V207L substitutions (vertical lettering) and foldon; the other figure labels are as described in FIG. 1A.

FIG. 2 Study design overview.

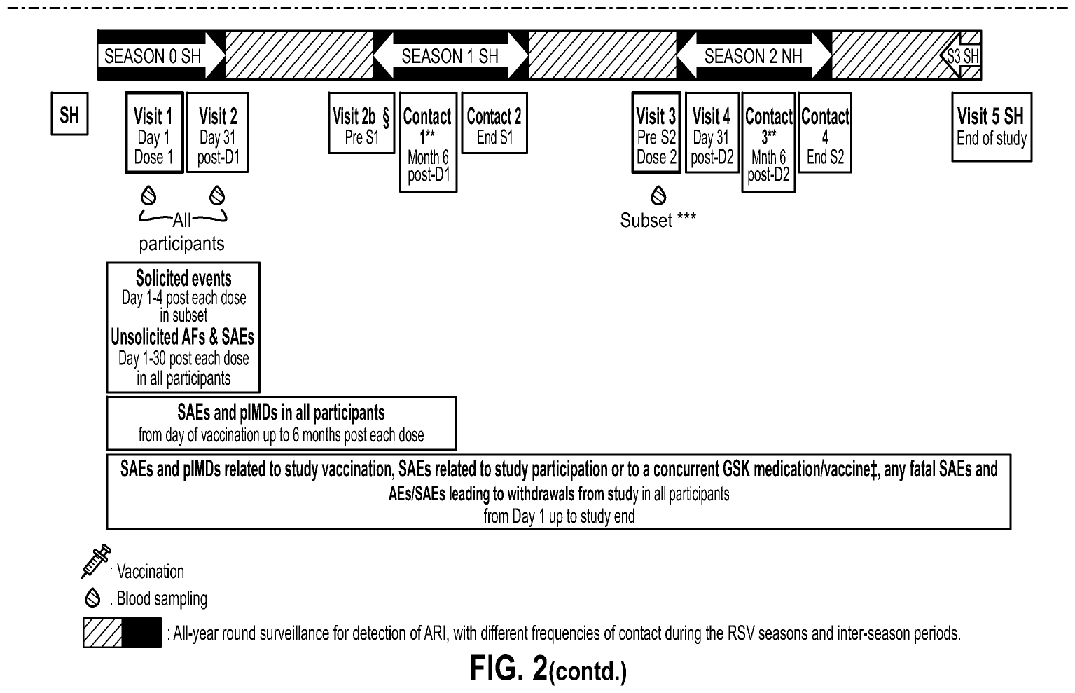

For simplicity, the randomization in this figure is presented as 1:1 between the RSVPreF3 OA vaccine and the placebo group. Participants are randomized with a ratio of 1:1:1:3 to 1 of 4 study groups (RSVPreF3 Lot 1/2/3 versus Placebo) for Part 1 of the study and a ratio of 1:1 to 1 of 2 study groups (RSVPreF3 Lot 4 versus Placebo) for Part 2 before Season 1.

### Key:

AE: adverse event; ARI: acute respiratory infection; NH: Northern hemisphere; SH: Southern hemisphere; pIMD: potential immune-mediated disease; RSV: respiratory syncytial virus; SAE: serious adverse event

### DETAILED DESCRIPTION OF THE INVENTION

The present invention relates to vaccination against respiratory syncytial virus (RSV), in particular to the use of a vaccine formulation comprising an RSV F fusion protein (RSV F protein) antigen and an adjuvant in methods of prevention of RSV infection and disease in older adults.

Compositions which may find use in accordance with the invention are useful for protection against or prevention of RSV disease. RSV disease includes acute respiratory infection (RSV ARI) and lower respiratory tract disease (RSV LRTD) which may be severe (severe RSV LRTD). In older adults with pre-existing medical conditions (“comorbidities”), RSV infection may lead to higher incidence of severe RSV LRTD, exacerbation of the underlying medical condition and/or increased rates of hospitalization. Older adults with underlying cardiorespiratory and endocrine or metabolic conditions (such as chronic obstructive pulmonary disease [COPD], congestive heart failure, and diabetes) in particular are at increased risk of RSV ARI. This population often has more severe clinical presentations that may require medical care and may lead to hospitalization, exacerbation of underlying conditions, or death.

In certain embodiments, there is provided a composition comprising a trimeric RSV F protein in the prefusion conformation and an adjuvant comprising a saponin and a TLR-4 agonist for use in a method of protection against, or prevention of, RSV disease in a human subject having a pre-existing medical condition. In certain embodiments, the pre-existing medical condition is a cardiorespiratory or endocrine/metabolic condition. In certain embodiments the composition comprising a trimeric RSV F protein in the prefusion conformation and an adjuvant comprising a saponin and a TLR-4 agonist finds use in a method of protection against, or prevention of, RSV disease in a human subject having a more than one, for example two pre-existing medical cardiorespiratory or endocrine/metabolic conditions such as chronic obstructive pulmonary disease (COPD) or asthma, coronary artery disease (CAD), congestive heart failure (CHF), diabetes or advanced renal or liver disease. In particular, the compositions may find use in a method of protection against, or prevention of RSV disease in a human subject having one or two pre-existing medical conditions selected from COPD, CAD, CHF or diabetes. In certain embodiments the subject has one or more, for example 1 or 2 pre-existing medical conditions selected from COPD, CAD, CHF or diabetes, for example COPD and CHF or COPD and diabetes, in particular embodiments the individual has diabetes.

In one embodiment, the composition is used in the prevention of the incidence of RSV disease. By prevention is meant the reduction of incidence of RSV disease. In one embodiment, the composition is used in the prevention of the incidence of RSV ARI. In another embodiment, the composition is used in the prevention of the incidence of RSV LRTD or severe RSV LRTD.

In one aspect, there is herein provided a method of protection against, or prevention of, RSV disease caused by RSV A and RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B.

In a clinical study, a composition comprising a trimeric RSV F protein in the prefusion conformation, derived from the F protein of the RSV A subtype, and an adjuvant comprising a saponin and a TLR-4 agonist, provided comparable protection against RSV disease caused by RSV A and against RSV disease caused by RSV B. Thus, there is provided a composition comprising a trimeric RSV F protein in the prefusion conformation derived from the F protein of the RSV A subtype and an adjuvant comprising a saponin and a TLR-4 agonist for use in a method of protecting a population from RSV disease caused by RSV A and RSV B. Accordingly, there is herein provided a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B for use in the prevention of RSV disease caused by RSV A and RSV B.

In particular embodiments, a composition is provided for use in the prevention of, or for providing protection against RSV disease caused by RSV A or by RSV B in an individual human subject, wherein the composition comprises a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B and wherein the individual is aged 60 years or older. Thus, at the population level, the compositions herein provided will provide protection against RSV disease caused by RSV A and RSV B. At an individual level, protection is provided against disease caused by RSV A or RSV B, or by disease caused by co-infection with RSV A and RSV B.

Accordingly, there is herein provided a method of protection against, or prevention of, RSV disease caused by RSV A and/or RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B. There is also provided a composition for use in protection against or prevention of RSV disease caused by RSV A and/or RSV B in a human subject, comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B.

### Pharmaceutical Compositions/Routes of Administration/Dosages

### Compositions

The compositions of use in the present invention comprise a trimeric RSV F protein in the prefusion conformation and an adjuvant. The compositions are formulated for intramuscular injection to a human subject. The adjuvant may be formulated separately from the protein composition, though for convenience is typically co-formulated. When the adjuvant is formulated separately from the immunogenic composition, for example as an aqueous liquid whilst the immunogenic composition is lyophilized, the liquid adjuvant may be utilized to reconstitute the lyophilized immunogenic composition for administration. The adjuvant comprises a saponin and a TLR-4 agonist, optionally in an aqueous liposomal formulation. A typical dose will contain 60-180 ug RSV F, for example 60, 80, 100, 120, 140 or 180 ug. Typically a composition for use in the present invention will contain 100-140 ug RSV F, such as 120 ug, delivered in a 0.5 mL or 1 ml dose.

The vaccination schedule may comprise several doses of the composition, however in one embodiment of the invention, a single dose of the composition is administered to the individual. In another embodiment, the vaccination schedule consists of 1 dose of the composition followed by a further dose approximately 1 year later. Thus, the vaccination may be seasonal, with administration approximately annually prior to the RSV season. Alternatively, following an initial vaccination, the duration of protection may be sufficient that the individual may receive a further administration at least 2, 3, 4, 5, 6, 7, 8, 9, 10, etc. years after the initial dose, for example the vaccination schedule may consist of 1 dose of the composition followed by a further dose approximately 2 years later or, in a further embodiment, approximately 3 years later. The composition is typically administered via the intramuscular route, although alternative routes may be considered, e.g. intradermal or subcutaneous.

The use or method of preventing RSV disease in accordance with the present invention (the vaccination) provides exceptionally high efficacies. In one embodiment, the efficacy of the vaccination is expressed as the reduction of the occurrence of RSV disease (such as RSV ARI, RSV LRTD or RSV severe LRTD) in a population after receiving the composition of the invention compared to placebo. The vaccination efficacy of reducing the occurrence of RSV disease in a population of adults aged 60 years or over compared to placebo is 60% or more, suitably 70% or more, suitably 80% or more, suitably 85% or more, suitably 90% or more. In particular embodiments, the efficacy is 80% or more. In certain embodiments the efficacy of reducing the occurrence of RSV B in a population of adults aged 60 years or over compared to placebo is comparable to the efficacy of reducing the occurrence of RSV A.

The use or method of preventing RSV disease in accordance with the present invention (the vaccination) provides exceptionally high efficacies in older adults. In certain embodiments, the vaccination efficacy of reducing the occurrence of RSV disease in a population of adults aged 70-79 years or over compared to placebo is 80% or more, suitably 85% or more, suitably 90% or more. In particular embodiments, the efficacy is 90% or more.

The use or method of preventing RSV disease in accordance with the present invention (the vaccination) provides exceptionally high efficacies in older adults with a pre-existing medical condition. In particular embodiments, the vaccination efficacy of reducing the occurrence of RSV disease in a population of adults aged 60 years or over with a pre-existing medical condition is as high or higher than in those without a pre-existing medical condition.

### RSV F Protein

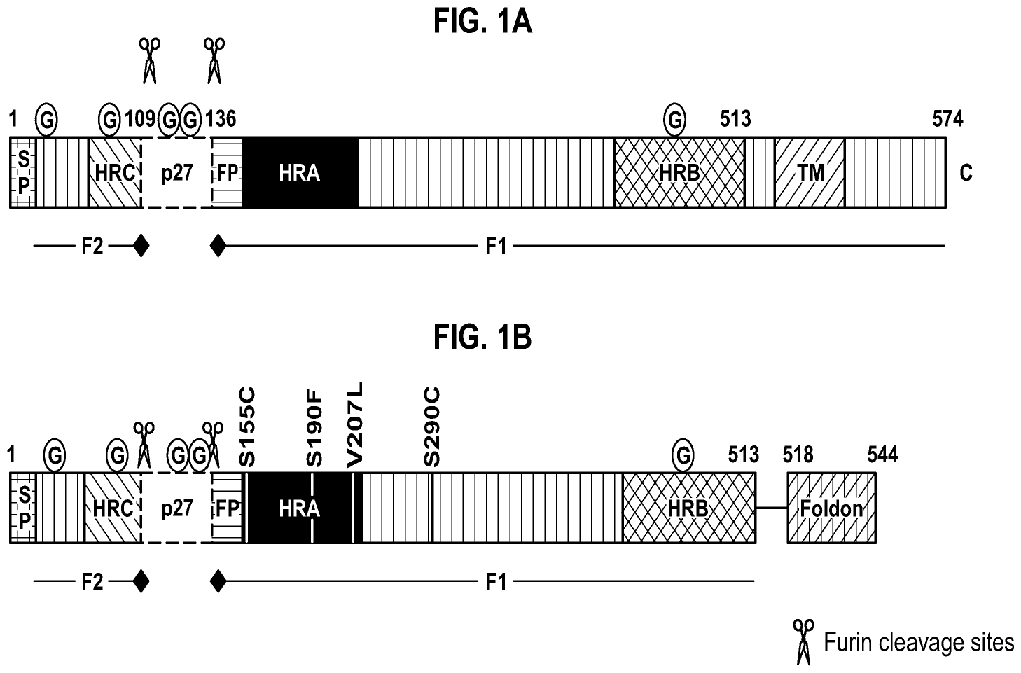

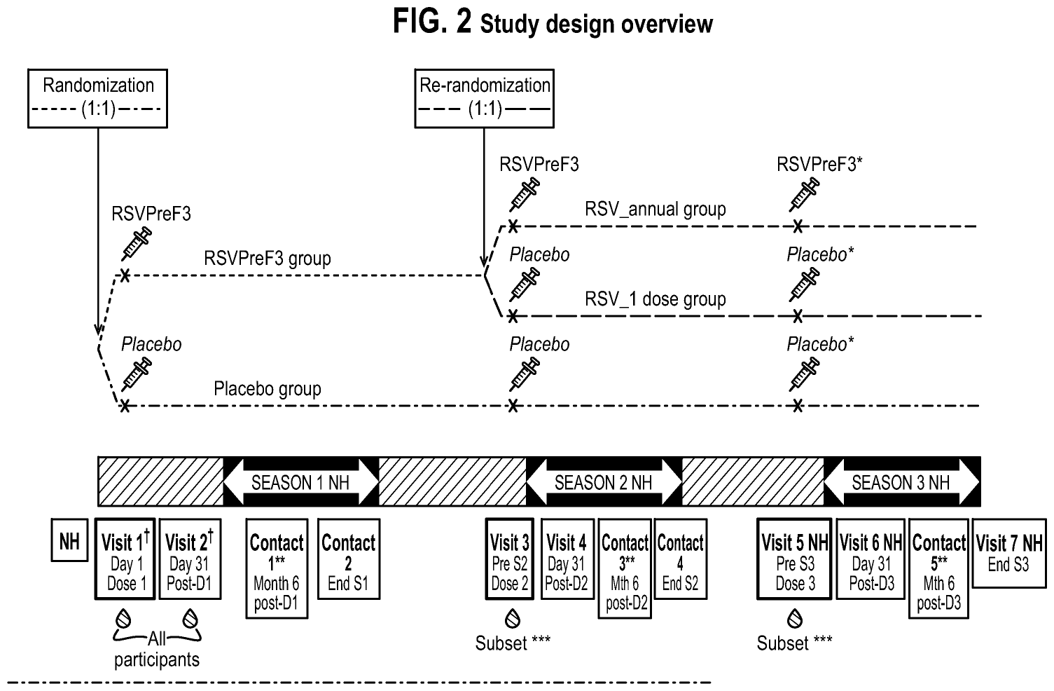

In a viral-infected cell, the RSV F protein is translated from mRNA into an approximately 574 amino acid protein designated F0 (FIG. 1A). Post-translational processing of F0 includes removal of an N-terminal signal peptide by a signal peptidase in the endoplasmic reticulum. F0 is also cleaved at two sites (approximately 109/110 and approximately 136/137) by cellular proteases (in particular furin) in the trans-Golgi. This cleavage results in the removal of a short intervening sequence (p27) and generates two subunits designated F1 (˜50 kDa; C-terminal; approximately residues 137-574) and F2 (˜20 kDa; N-terminal; approximately residues 1-109) that remain associated with each other via a disulphide bond. These covalently linked F2 and F1 domains are, together, the RSV F protein monomer which trimerises to form the mature F protein (also known as the F protein homotrimer or the trimeric F protein). The F1 portion of the monomer contains a hydrophobic fusion peptide (FP) at its N-terminus and also two amphipathic heptad-repeat regions (HRA and HRB). HRA is near the fusion peptide (FP) and HRB is near the transmembrane domain (TM). Three F1-F2 monomers are assembled as homotrimers of F1-F2 in the virion and are embedded in the viral membrane via the transmembrane domain. It is this trimer of heterodimers which is referred to as the trimeric RSV protein.

The RSV F protein initially folds into a metastable “prefusion” conformation. During cell entry (infection) the viral membrane and the cell membrane fuse to form the infection pore. This process is mediated by the F protein. During this process, the prefusion conformation undergoes refolding and conformational changes to its stable “postfusion” conformation. In compositions of use in the present invention, the RSV F protein is in prefusion conformation. The epitopes of the prefusion conformation may be better able to elicit antibodies that can recognize and neutralize natural virions.

In compositions of use in the present invention, the trimeric RSV protein is a recombinant protein, stabilized in the prefusion conformation of the naturally occurring F protein. For use in vaccine compositions, the RSV F protein is desirably soluble, rather than membrane-bound. Thus, the trimeric RSV F protein in the prefusion conformation may have the transmembrane and cytoplasmic domains of the wild type protein replaced by a heterologous trimerization domain. The trimeric RSV F protein in the prefusion conformation may be a recombinant trimeric protein in which each monomer has (i) at least two amino acid substitutions relative to the wild type RSV F protein; and (ii) addition of a heterologous trimerization domain replacing the transmembrane and cytoplasmic domain of RSV F. In certain embodiments, the at least two amino acid substitutions relative to the wild type RSV F protein include the introduction of cysteine residues leading to the formation of a non-natural disulfide bond. Further modifications can include amino acid substitutions to fill hydrophobic cavities in the molecule. It has been shown that most of the RSV neutralizing activity present in serum from previously infected individuals is directed to the prefusion conformation of RSV F protein.

The trimeric RSV protein in prefusion conformation used in the example (referred to as RSVPreF3), the amino acid sequence of which is given in SEQ ID NO:2, elicited higher levels of NAbs in animal models than those observed with an RSV F protein in the postfusion conformation of the naturally occurring F protein [Steff A M, Monroe J, Friedrich K, et al. (2017) Pre-fusion RSV F strongly boosts pre-fusion specific neutralizing responses in cattle pre-exposed to bovine RSV. Nat Commun. 2017; 8:1085].

### Adjuvants

The immunogenic composition for use in the present invention is formulated for intramuscular administration with an adjuvant including a TLR 4 agonist and a saponin, optionally in a liposomal formulation.

An adjuvant of interest comprises a TLR4 agonist and a saponin in liposomal formulation.

A suitable example of a TLR4 agonist is a lipopolysaccharide, suitably a non-toxic derivative of lipid A, particularly a monophosphoryl lipid A and more particularly 3-de-O-acylated monophosphoryl lipid A (3D-MPL).

3D-MPL can be produced according to the methods described in published patent application GB 2 220 211 A. Chemically it is a mixture of 3-deacylated monophosphoryl lipid A with 4, 5 or 6 acylated chains. In the context of the present invention, small particle 3D-MPL may be used to prepare the aqueous adjuvant composition. Small particle 3D-MPL has a particle size such that it may be sterile-filtered through a 0.22 μm filter. Such preparations are described in published patent application WO94/21292.

Other TLR4 agonists which can be used are alkyl glucosaminide phosphates (AGPs) such as those described in WO98/50399 or U.S. Pat. No. 6,303,347 (processes for preparation of AGPs are also described). Some AGPs are TLR4 agonists, and some are TLR4 antagonists.

Other TLR4 agonists which may be of use in the immunogenic compositions described herein include Glucopyranosyl Lipid Adjuvant (GLA) such as described in WO2008/153541 or WO2009/143457 or the literature articles Coler R N et al. (2011) Development and Characterization of Synthetic Glucopyranosyl Lipid Adjuvant System as a Vaccine Adjuvant. PLOS ONE 6(1): e16333. doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLOS ONE 7(7): e41144. doi:10.1371/journal.pone.0041144. A suitable saponin for use in the present invention is Quil A and its derivatives. Quil A is a saponin preparation isolated from the South American tree Quillaja saponaria Molina and was first described as having adjuvant activity by Dalsgaard et al. in 1974 (“Saponin adjuvants”, Archiv. für die gesamte Virusforschung, Vol. 44, Springer Verlag, Berlin, p243-254). Purified fractions of Quil A have been isolated by HPLC which retain adjuvant activity without the toxicity associated with Quil A (see, for example, published patent application EP0362278). Fractions of general interest include QS7, QS17, QS18 and QS21, for example QS7 and QS21 (also known as QA7 and QA21). QS21 is a saponin of particular interest.

The saponin may conveniently be presented in a composition where it is quenched with an exogenous sterol, such as cholesterol. Suitably the ratio of saponin:sterol (e.g. QS21: cholesterol) is from 1:100 to 1:1 w/w, such as from 1:10 to 1:1 w/w, e.g. from 1:5 to 1:1 w/w.

In a liposomal adjuvant formulation, liposome size may vary from 30 nm to several um depending on the phospholipid composition and the method used for preparation. In the present invention, the liposome size will be in the range of 50 nm to 200 nm, especially 60 nm to 180 nm, such as 70-165 nm. Optimally, the liposomes should be stable and have a diameter of ˜100 nm to allow convenient sterilization by filtration.

The liposomes may be formed from many different lipids, although desirably contain DOPC (1,2-dioleoyl-sn-glycero-3-phosphocholine), or, consist essentially of DOPC and sterol (with saponin and TLR4 agonist as appropriate).

Combinations of different adjuvants, such as those mentioned hereinabove, can also be used in compositions with a RSV F protein. For example, as already noted, QS21 can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL: QS21.

In particular embodiments, the adjuvant comprises QS21 and 3D-MPL in the same final concentration per human dose. In one aspect of this embodiment, a human dose of the compositions described herein comprises a final level of 50 μg of 3D-MPL and 50 μg of QS21. In a further embodiment, a human dose comprises a final level of 25 μg of 3D-MPL and 25 μg of QS21.

In a further embodiment, a human dose comprises a final level of 10 μg each of MPL and QS21. In a further specific embodiment is provided an adjuvant composition having a volume of 500 μl or 250 μl and comprising a level of 25 μg of 3D-MPL and 25 μg of QS21, or 10 μg each of MPL and QS21. A formulation of QS21 and 3D-MPL in a 1:1 ratio with cholesterol and DOPC in a liposome (AS01) may be used in a dose containing 50 μg of 3D-MPL and 50 μg of QS21 (AS01_{B}) or 25 μg of 3D-MPL and 25 μg of QS21 (AS01_{E}).

Accordingly, there is herein provided a method of protection against, or prevention of, RSV disease caused by RSV A and/or RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B, wherein the trimeric RSV F protein in the prefusion conformation is present in an amount of 60-180 μg, for example 120 μg and wherein the adjuvant comprises 50 μg or 25 μg of 3D-MPL and 50 μg or 25 μg of QS21, DOPC and cholesterol in a liposome. Further provided herein is a composition comprising a trimeric RSV F protein in the prefusion conformation and an adjuvant comprising a saponin and a TLR-4 agonist for use in protection against or prevention of RSV disease, wherein the trimeric RSV F protein in the prefusion conformation is derived from RSV A and the composition does not contain an RSV F protein derived from RSV B, wherein the trimeric RSV F protein in the prefusion conformation is present in an amount of 60-180 μg, for example 120 ug and wherein the adjuvant comprises 25 μg of 3D-MPL and 25 μg of QS21, DOPC and cholesterol in a liposome.

### General Manufacturing Methods

Trimeric RSV F protein in the prefusion conformation may be expressed as a soluble and secreted protein in a suitable, for example mammalian, cell line. Suitable mammalian cells include, for example, Chinese hamster ovary (CHO) cells, human embryonic kidney cells (HEK293) cells and the like. Expressed, soluble, protein may be purified from the cell culture using any suitable methods. For example, methods for purifying expressed polypeptides by immunoaffinity chromatography are known in the art. Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the RSV F protein can be engineered to include a “tag” that facilitates purification, such as an epitope tag or a HIS tag. Such tagged polypeptides can conveniently be purified, for example from conditioned media, by chelating chromatography or affinity chromatography.

### EMBODIMENTS OF THE INVENTION

1. A composition comprising a trimeric RSV F protein in the prefusion conformation and an adjuvant comprising a saponin and a TLR-4 agonist for use in a method of protection against, or prevention of, RSV disease in a human subject of 60 years of age or older.
2. The composition for use as defined in embodiment 1, wherein the subject is 65 years of age or older, 70 years of age or older, or 75 years of age or older.
3. The composition for use as defined in embodiment 1 or embodiment 2 wherein the subject is 80 years of age or older.
4. The composition for use as defined in any one of embodiments 1-3, wherein the saponin is selected from Quil A and its derivatives, for example is selected from QS7, QS17, QS18 and QS21 fractions of Quil A.
5. The composition for use as defined in any one of embodiments 1-4, wherein the TLR-4 agonist is a lipopolysaccharide, for example is a monophosphoryl lipid A, for example 3-de-O-acylated monophosphoryl lipid A (3D-MPL).
6. The composition for use as defined in any one of embodiments 1-5 wherein the adjuvant further comprises liposomes.
7. The composition for use as defined in embodiment 6 where in the liposomes comprise DOPC.
8. The composition for use as defined in any one of embodiments 1-7 wherein the adjuvant comprises QS21, cholesterol, 3D-MPL and liposomes containing DOPC.
9. The composition for use as defined in any one of embodiments 1-8 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
10. The composition for use as defined in embodiment 9 wherein the at least two amino acid substitutions comprise the introduction of two cysteine residues and an artificial disulphide bond is formed between the introduced cysteine residues.
11. The composition for use as defined in embodiment 9 or embodiment 10 wherein the heterologous trimerization domain is a foldon domain of bacteriophage T4 fibritin.
12. The composition for use as defined in any one of embodiments 9-11 wherein the trimeric RSV F protein in the prefusion conformation is a recombinant trimeric protein having S155C, S290C, S190F, and V207L substitutions relative to the sequence of SEQ ID NO:1.
13. The composition for use as defined in any one of embodiments 9-12 wherein each monomer of the trimeric RSV F protein in the prefusion conformation has the amino acid sequence of SEQ ID NO:2.
14. A method of protection against, or prevention of, RSV disease in a human subject of 60 years of age or older, comprising the step of administering to the subject a composition comprising a trimeric RSV F protein in the prefusion conformation and an adjuvant comprising a saponin and a TLR-4 agonist.
15. The method as defined in embodiment 14, wherein the subject is 65 years of age or older, 70 years of age or older, or 75 years of age or older.
16. The method as defined in embodiment 14 or embodiment 15 wherein the subject is 80 years of age or older.
17. The method as defined in any one of embodiments 14-16 wherein the saponin is selected from Quil A and its derivatives, for example is selected from QS7, QS17, QS18 and QS21 fractions of Quil A.
18. The method as defined in any one of embodiments 14-17, wherein the TLR-4 agonist is a lipopolysaccharide, for example is a monophosphoryl lipid A, for example 3-de-O-acylated monophosphoryl lipid A (3D-MPL).
19. The method as defined in any one of embodiments 14-18 wherein the adjuvant further comprises liposomes.
20. The method as defined in embodiment 19 wherein the liposomes comprise DOPC.
21. The method as defined in any one of embodiments 14-20 wherein the adjuvant comprises QS21, cholesterol, 3D-MPL and liposomes containing DOPC.
22. The method as defined in any one of embodiments 14-21 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
23. The method as defined in embodiment 22 wherein the at least two amino acid substitutions comprise the introduction of two cysteine residues and an artificial disulphide bond is formed between the introduced cysteine residues.
24. The method as defined in embodiment 22 or embodiment 23 wherein the heterologous trimerization domain is a foldon domain of bacteriophage T4 fibritin.
25. The method as defined in any one of embodiments 22-24 wherein the trimeric RSV F protein in the prefusion conformation is a recombinant trimeric protein having S155C, S290C, S190F, and V207L substitutions relative to the sequence of SEQ ID NO:1.
26. The method as defined in any one of embodiments 22-25 wherein each monomer of the trimeric RSV F protein in the prefusion conformation has the amino acid sequence of SEQ ID NO:2.
27. A method for reducing the incidence of RSV disease in a human subject of 60 years of age or older, comprising the step of administering to the subject a composition comprising a trimeric RSV F protein in the prefusion conformation and an adjuvant comprising a saponin and a TLR-4 agonist.
28. The method as defined in embodiment 27, wherein the subject is 65 years of age or older, 70 years of age or older, or 75 years of age or older.
29. The method as defined in embodiment 27 or embodiment 28 wherein the subject is 80 years of age or older.
30. The method as defined in any one of embodiments 27-29, wherein the saponin is selected from Quil A and its derivatives, for example is selected from QS7, QS17, QS18 and QS21 fractions of Quil A.
31. The method as defined in any one of embodiments 27-30, wherein the TLR-4 agonist is a lipopolysaccharide, for example is a monophosphoryl lipid A, for example 3-de-O-acylated monophosphoryl lipid A (3D-MPL).
32. The method as defined in any one of embodiments 27-31 wherein the adjuvant further comprises liposomes.
33. The method as defined in embodiment 32 where in the liposomes comprise DOPC.
34. The method as defined in any one of embodiments 27-33 wherein the adjuvant comprises QS21, cholesterol, 3D-MPL and liposomes containing DOPC.
35. The method as defined in any one of embodiments 27-34 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
36. The method as defined in embodiment 35 wherein the at least two amino acid substitutions comprise the introduction of two cysteine residues and an artificial disulphide bond is formed between the introduced cysteine residues.
37. The method as defined in embodiment 35 or embodiment 36 wherein the heterologous trimerization domain is a foldon domain of bacteriophage T4 fibritin.
38. The method as defined in any one of embodiments 35-37 wherein the trimeric RSV F protein in the prefusion conformation is a recombinant trimeric protein having S155C, S290C, S190F, and V207L substitutions relative to the sequence of SEQ ID NO:1.
39. The method as defined in any one of embodiments 35-38 wherein each monomer of the trimeric RSV F protein in the prefusion conformation has the amino acid sequence of SEQ ID NO:2.
40. A composition comprising a trimeric RSV F protein in the prefusion conformation and an adjuvant comprising a saponin and a TLR-4 agonist for use in a method for the prevention of RSV disease in a human subject of 60 years of age or older.
41. The composition for use as defined in embodiment 40, wherein the subject is 65 years of age or older, 70 years of age or older, or 75 years of age or older.
42. The composition for use as defined in embodiment 40 or embodiment 41 wherein the subject is 80 years of age or older.
43. The composition as defined in any one of embodiments 40-42, wherein the saponin is selected from Quil A and its derivatives, for example is selected from QS7, QS17, QS18 and QS21 fractions of Quil A.
44. The composition for use as defined in any one of embodiments 40-43, wherein the TLR-4 agonist is a lipopolysaccharide, for example is a monophosphoryl lipid A, for example 3-de-O-acylated monophosphoryl lipid A (3D-MPL).
45. The composition for use as defined in any one of embodiments 40-44 wherein the adjuvant further comprises liposomes.
46. The composition for use as defined in embodiment 45 where in the liposomes comprise DOPC.
47. The composition for use as defined in any one of embodiments 40-46 wherein the adjuvant comprises QS21, cholesterol, 3D-MPL and liposomes containing DOPC.
48. The composition for use as defined in any one of embodiments 40-47 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
49. The composition for use as defined in embodiment 48 wherein the at least two amino acid substitutions comprise the introduction of two cysteine residues and an artificial disulphide bond is formed between the introduced cysteine residues.
50. The composition for use as defined in embodiment 48 or embodiment 49 wherein the heterologous trimerization domain is a foldon domain of bacteriophage T4 fibritin.
51. The composition for use as defined in any one of embodiments 48-50 wherein the trimeric RSV F protein in the prefusion conformation is a recombinant trimeric protein having S155C, S290C, S190F, and V207L substitutions relative to the sequence of SEQ ID NO:1.
52. The composition for use as defined in any one of embodiments 48-51 wherein each monomer of the trimeric RSV F protein in the prefusion conformation has the amino acid sequence of SEQ ID NO:2.
53. A composition comprising a trimeric RSV F protein in the prefusion conformation wherein each monomer of the trimeric RSV F protein in the prefusion conformation has the amino acid sequence of SEQ ID NO:2, and an adjuvant comprising QS21, cholesterol, 3D-MPL and liposomes containing DOPC, for use in a method for the prevention of RSV disease in a human subject of 60 years of age or older.
54. A method of protection against, or prevention of, RSV disease caused by RSV A and RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B.
55. A method for the prevention of RSV disease caused by RSV A and RSV B in a human subject, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B.
56. The method as defined in embodiment 54 or embodiment 55, wherein the subject is 60 years of age or older, 65 years of age or older, 70 years of age or older, or 75 years of age or older.
57. The method as defined in in any one of embodiments 54-56, wherein the subject is 80 years of age or older.
58. The method as defined in any one of embodiments 54-57, wherein the saponin is selected from Quil A and its derivatives, for example is selected from QS7, QS17, QS18 and QS21 fractions of Quil A.
59. The method as defined in any one of embodiments 54-58, wherein the TLR-4 agonist is a lipopolysaccharide, for example is a monophosphoryl lipid A, for example 3-de-O-acylated monophosphoryl lipid A (3D-MPL).
60. The method as defined in any one of embodiments 54-59 wherein the adjuvant further comprises liposomes.
61. The method as defined in embodiment 60 where in the liposomes comprise DOPC.
62. The method as defined in any one of embodiments 54-61 wherein the adjuvant comprises QS21, cholesterol, 3D-MPL and liposomes containing DOPC.
63. The method as defined in any one of embodiments 54-62 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
64. The method as defined in embodiment 63 wherein the at least two amino acid substitutions comprise the introduction of two cysteine residues and an artificial disulphide bond is formed between the introduced cysteine residues.
65. The method as defined in embodiment 63 or embodiment 64 wherein the heterologous trimerization domain is a foldon domain of bacteriophage T4 fibritin.
66. The method as defined in any one of embodiments 63-65 wherein the trimeric RSV F protein in the prefusion conformation is a recombinant trimeric protein having S155C, S290C, S190F, and V207L substitutions relative to the sequence of SEQ ID NO:1.
67. The method as defined in any one of embodiments 63-66 wherein each monomer of the trimeric RSV F protein in the prefusion conformation has the amino acid sequence of SEQ ID NO:2.
68. A method for the prevention of RSV disease caused by RSV A and RSV B in a human subject, comprising the step of administration to the subject of a composition comprising a trimeric RSV F protein in the prefusion conformation wherein each monomer of the trimeric RSV F protein in the prefusion conformation has the amino acid sequence of SEQ ID NO:2, and an adjuvant comprising QS21, cholesterol, 3D-MPL and liposomes containing DOPC, wherein the trimeric RSV F protein in the prefusion conformation is derived from RSV A and the composition does not contain an RSV F protein derived from RSV B.
69. A composition comprising a trimeric RSV F protein in the prefusion conformation derived from RSV A and an adjuvant comprising a saponin and a TLR-4 agonist for use in a method for the prevention of RSV disease caused by RSV A and RSV B in a human subject, wherein the composition does not contain an RSV F protein derived from RSV B.
70. The composition for use as defined in embodiment 69, wherein the subject is 60 years of age or older, 65 years of age or older, 70 years of age or older, or 75 years of age or older.
71. The composition for use as defined in embodiment 69 or embodiment 70 wherein the subject is 80 years of age or older.
72. The composition for use as defined in any one of embodiments 69-71, wherein the saponin is selected from Quil A and its derivatives, for example is selected from QS7, QS17, QS18 and QS21 fractions of Quil A.
73. The composition for use as defined in any one of embodiments 69-72, wherein the TLR-4 agonist is a lipopolysaccharide, for example is a monophosphoryl lipid A, for example 3-de-O-acylated monophosphoryl lipid A (3D-MPL).
74. The composition for use as defined in any one of embodiments 69-73 wherein the adjuvant further comprises liposomes.
75. The composition for use as defined in embodiment 74 where in the liposomes comprise DOPC.
76. The composition for use as defined in any one of embodiments 69-75 wherein the adjuvant comprises QS21, cholesterol, 3D-MPL and liposomes containing DOPC.
77. The composition for use as defined in any one of embodiments 69-76 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
78. The composition for use as defined in embodiment 77 wherein the at least two amino acid substitutions comprise the introduction of two cysteine residues and an artificial disulphide bond is formed between the introduced cysteine residues.
79. The composition for use as defined in embodiment 77 or embodiment 78 wherein the heterologous trimerization domain is a foldon domain of bacteriophage T4 fibritin.
80. The composition for use as defined in any one of embodiments 77-79 wherein the trimeric RSV F protein in the prefusion conformation is a recombinant trimeric protein having S155C, S290C, S190F, and V207L substitutions relative to the sequence of SEQ ID NO:1.
81. The composition for use as defined in any one of embodiments 77-80 wherein each monomer of the trimeric RSV F protein in the prefusion conformation has the amino acid sequence of SEQ ID NO:2.
82. A composition comprising a trimeric RSV F protein in the prefusion conformation derived from RSV A, wherein each monomer of the trimeric RSV F protein in the prefusion conformation has the amino acid sequence of SEQ ID NO:2, and an adjuvant comprising QS21, cholesterol, 3D-MPL and liposomes containing DOPC, for use in a method for the prevention of RSV disease caused by RSV A and RSV B in a human subject, wherein the composition does not contain an RSV F protein derived from RSV B.
83. A composition comprising a trimeric RSV F protein in the prefusion conformation and an adjuvant comprising a saponin and a TLR-4 agonist, for use in a method for the prevention of RSV disease in a human subject of 60 years of age or older having a pre-existing medical condition.
84. The composition for use as defined in embodiment 83 wherein the pre-existing medical condition is a cardiovascular, respiratory or endocrine condition.
85. The composition for use as defined in embodiment 83, wherein the pre-existing medical condition is a chronic cardiovascular or pulmonary disease, diabetes mellitus type 1 or type 2, chronic heart failure, advanced liver disease or advanced renal disease.
86. The composition for use as defined in embodiment 83 wherein the pre-existing medical condition is COPD (chronic obstructive pulmonary disease), asthma or hypertension.
87. The composition for use as defined in any one of embodiments 83-86, wherein the subject is 65 years of age or older, 70 years of age or older, or 75 years of age or older.
88. The composition for use as defined in any one of embodiments 83-87 wherein the subject is 80 years of age or older.
89. The composition for use as defined in any one of embodiments 83-88, wherein the saponin is selected from Quil A and its derivatives, for example is selected from QS7, QS17, QS18 and QS21 fractions of Quil A.
90. The composition for use as defined in any one of embodiments 83-89, wherein the TLR-4 agonist is a lipopolysaccharide, for example is a monophosphoryl lipid A, for example 3-de-O-acylated monophosphoryl lipid A (3D-MPL).
91. The composition for use as defined in any one of embodiments 83-90 wherein the adjuvant further comprises liposomes.
92. The composition for use as defined in embodiment 91 where in the liposomes comprise DOPC.
93. The composition for use as defined in any one of embodiments 83-92 wherein the adjuvant comprises QS21, cholesterol, 3D-MPL and liposomes containing DOPC.
94. The composition for use as defined in any one of embodiments 83-93 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
95. The composition for use as defined in embodiment 94 wherein the at least two amino acid substitutions comprise the introduction of two cysteine residues and an artificial disulphide bond is formed between the introduced cysteine residues.
96. The composition for use as defined in embodiment 94 or embodiment 95 wherein the heterologous trimerization domain is a foldon domain of bacteriophage T4 fibritin.
97. The composition for use as defined in any one of embodiments 94-96 wherein the trimeric RSV F protein in the prefusion conformation is a recombinant trimeric protein having S155C, S290C, S190F, and V207L substitutions relative to the sequence of SEQ ID NO:1.
98. The composition for use as defined in any one of embodiments 94-97 wherein each monomer of the trimeric RSV F protein in the prefusion conformation has the amino acid sequence of SEQ ID NO:2.
99. A composition comprising a trimeric RSV F protein in the prefusion conformation wherein each monomer of the trimeric RSV F protein in the prefusion conformation has the amino acid sequence of SEQ ID NO:2, and an adjuvant comprising QS21, cholesterol, 3D-MPL and liposomes containing DOPC, for use in a method for the prevention of RSV disease in a human subject of 60 years of age or older having a pre-existing medical condition selected from a chronic cardiovascular or pulmonary disease, diabetes mellitus type 1 or type 2, chronic heart failure, advanced liver disease, advanced renal disease COPD, asthma or hypertension.
100. A method for protection against, or prevention of, RSV disease in a human subject of 60 years of age or older having a pre-existing medical condition comprising the step of administration to the subject of a composition comprising a trimeric RSV F protein in the prefusion conformation and an adjuvant comprising a saponin and a TLR-4 agonist.
101. A method for the prevention of RSV disease in a human subject of 60 years of age or older having a pre-existing medical condition comprising the step of administration to the subject of a composition comprising a trimeric RSV F protein in the prefusion conformation and an adjuvant comprising a saponin and a TLR-4 agonist.
102. The method as defined in embodiment 100 or embodiment 101 wherein the pre-existing medical condition is a cardiovascular, respiratory or endocrine condition.
103. The method as defined in embodiment 100 or embodiment 101, wherein the pre-existing medical condition is a chronic cardiovascular or pulmonary disease, diabetes mellitus type 1 or type 2, chronic heart failure, advanced liver disease or advanced renal disease.
104. The method as defined in embodiment 100 or embodiment 101, wherein the pre-existing medical condition is COPD (chronic obstructive pulmonary disease), asthma or hypertension.
105. The method as defined in any one of embodiments 100-104, wherein the subject is 65 years of age or older, 70 years of age or older, or 75 years of age or older.
106. The method as defined in any one of embodiments 100-104 wherein the subject is 80 years of age or older.
107. The method as defined in any one of embodiments 100-106, wherein the saponin is selected from Quil A and its derivatives, for example is selected from QS7, QS17, QS18 and QS21 fractions of Quil A.
108. The method as defined in any one of embodiments 100-107, wherein the TLR-4 agonist is a lipopolysaccharide, for example is a monophosphoryl lipid A, for example 3-de-O-acylated monophosphoryl lipid A (3D-MPL).
109. The method as defined in any one of embodiments 100-108 wherein the adjuvant further comprises liposomes.
110. The method as defined in embodiment 109 where in the liposomes comprise DOPC. 111 The method as defined in any one of embodiments 100-110 wherein the adjuvant comprises QS21, cholesterol, 3D-MPL and liposomes containing DOPC.
112. The method as defined in any one of embodiments 100-111 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
113. The method as defined in embodiment 112 wherein the at least two amino acid substitutions comprise the introduction of two cysteine residues and an artificial disulphide bond is formed between the introduced cysteine residues.
114. The method as defined in embodiment 112 or embodiment 113 wherein the heterologous trimerization domain is a foldon domain of bacteriophage T4 fibritin.
115. The method as defined in any one of embodiments 112-114 wherein the trimeric RSV F protein in the prefusion conformation is a recombinant trimeric protein having S155C, S290C, S190F, and V207L substitutions relative to the sequence of SEQ ID NO:1.
116. The method as defined in any one of embodiments 112-115 wherein each monomer of the trimeric RSV F protein in the prefusion conformation has the amino acid sequence of SEQ ID NO:2.
117. A method for the prevention of RSV disease in a human subject of 60 years of age or older having a pre-existing medical condition selected from a chronic cardiovascular or pulmonary disease, diabetes mellitus type 1 or type 2, chronic heart failure, advanced liver disease, advanced renal disease COPD, asthma or hypertension, comprising the step of administration to the subject of a composition comprising a trimeric RSV F protein in the prefusion conformation wherein each monomer of the trimeric RSV F protein in the prefusion conformation has the amino acid sequence of SEQ ID NO:2, and an adjuvant comprising QS21, cholesterol, 3D-MPL and liposomes containing DOPC.
118. A composition comprising a trimeric RSV F protein in the prefusion conformation and an adjuvant comprising a saponin and a TLR-4 agonist for use in a method for the prevention of RSV disease in a human subject of 60 years of age or older, wherein the RSV disease is RSV acute respiratory infection (ARI), RSV lower respiratory tract disease (LRTD) or severe lower respiratory tract disease (severe LRTD).
119. The composition for use as defined in embodiment 118, wherein the subject is 65 years of age or older, 70 years of age or older, or 75 years of age or older.
120. The composition for use as defined in embodiment 118 or embodiment 119 wherein the subject is 80 years of age or older.
121. The composition for use as defined in any one of embodiments 118-120, wherein the saponin is selected from Quil A and its derivatives, for example is selected from QS7, QS17, QS18 and QS21 fractions of Quil A.
122. The composition for use as defined in any one of embodiments 118-121, wherein the TLR-4 agonist is a lipopolysaccharide, for example is a monophosphoryl lipid A, for example 3-de-O-acylated monophosphoryl lipid A (3D-MPL).
123. The composition for use as defined in any one of embodiments 118-122 wherein the adjuvant further comprises liposomes.
124. The composition for use as defined in embodiment 123 where in the liposomes comprise DOPC.
125. The composition for use as defined in any one of embodiments 118-124 wherein the adjuvant comprises QS21, cholesterol, 3D-MPL and liposomes containing DOPC.
126. The composition for use as defined in any one of embodiments 118-125 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
127. The composition for use as defined in embodiment 126 wherein the at least two amino acid substitutions comprise the introduction of two cysteine residues and an artificial disulphide bond is formed between the introduced cysteine residues.
128. The composition for use as defined in embodiment 126 or embodiment 127 wherein the heterologous trimerization domain is a foldon domain of bacteriophage T4 fibritin.
129. The composition for use as defined in any one of embodiments 126-128 wherein the trimeric RSV F protein in the prefusion conformation is a recombinant trimeric protein having S155C, S290C, S190F, and V207L substitutions relative to the sequence of SEQ ID NO:1.
130. The composition for use as defined in any one of embodiments 126-129 wherein each monomer of the trimeric RSV F protein in the prefusion conformation has the amino acid sequence of SEQ ID NO:2.
131. A composition comprising a trimeric RSV F protein in the prefusion conformation, wherein each monomer of the trimeric RSV F protein in the prefusion conformation has the amino acid sequence of SEQ ID NO:2, and an adjuvant comprising QS21, cholesterol, 3D-MPL and liposomes containing DOPC, for use in a method for the prevention of RSV disease in a human subject of 60 years of age or older, wherein the RSV disease is RSV acute respiratory infection (ARI), RSV lower respiratory tract disease (LRTD) or severe lower respiratory tract disease (severe LRTD).
132. A method for the prevention of RSV acute respiratory infection (ARI), RSV lower respiratory tract disease (LRTD) or RSV severe lower respiratory tract disease (RSV severe LRTD) in a human subject of 60 years of age or older comprising the step of administering to the subject a composition comprising a trimeric RSV F protein in the prefusion conformation and an adjuvant comprising a saponin and a TLR-4 agonist.
133. The method as defined in embodiment 132, wherein the subject is 65 years of age or older, 70 years of age or older, or 75 years of age or older.
134. The method as defined in embodiment 132 or embodiment 133 wherein the subject is 80 years of age or older.
135. The method as defined in any one of embodiments 132-134, wherein the saponin is selected from Quil A and its derivatives, for example is selected from QS7, QS17, QS18 and QS21 fractions of Quil A.
136. The method as defined in any one of embodiments 132-135, wherein the TLR-4 agonist is a lipopolysaccharide, for example is a monophosphoryl lipid A, for example 3-de-O-acylated monophosphoryl lipid A (3D-MPL).
137. The method as defined in any one of embodiments 132-136 wherein the adjuvant further comprises liposomes.
138. The method as defined in embodiment 137 where in the liposomes comprise DOPC.
139. The method as defined in any one of embodiments 132-138 wherein the adjuvant comprises QS21, cholesterol, 3D-MPL and liposomes containing DOPC.
140. The method as defined in any one of embodiments 132-139 wherein the trimeric RSV F protein in the prefusion conformation is a recombinant trimeric protein in which each monomer has (i) at least two amino acid substitutions relative to the wild type RSV F protein of RSV A of SEQ ID NO:1; and (ii) addition of a heterologous trimerization domain replacing the transmembrane and cytoplasmic domain of RSV F.
141. The method as defined in embodiment 140 wherein the at least two amino acid substitutions comprise the introduction of two cysteine residues and an artificial disulphide bond is formed between the introduced cysteine residues.
142. The method as defined in embodiment 140 or embodiment 141 wherein the heterologous trimerization domain is a foldon domain of bacteriophage T4 fibritin.
143. The method as defined in any one of embodiments 140-142 wherein the trimeric RSV F protein in the prefusion conformation is a recombinant trimeric protein having S155C, S290C, S190F, and V207L substitutions relative to the sequence of SEQ ID NO:1.
144. The method as defined in any one of embodiments 140-143 wherein each monomer of the trimeric RSV F protein in the prefusion conformation has the amino acid sequence of SEQ ID NO:2.
145. A method for the prevention of RSV acute respiratory infection (ARI), RSV lower respiratory tract disease (LRTD) or RSV severe lower respiratory tract disease (RSV severe LRTD) in a human subject of 60 years of age or older comprising the step of administering to the subject a composition comprising a trimeric RSV F protein in the prefusion conformation, wherein each monomer of the trimeric RSV F protein in the prefusion conformation has the amino acid sequence of SEQ ID NO:2, and an adjuvant comprising QS21, cholesterol, 3D-MPL and liposomes containing DOPC.
146. The use of a composition comprising a trimeric RSV F protein in the prefusion conformation and an adjuvant comprising a saponin and a TLR-4 agonist in the manufacture of a medicament for the prevention of RSV disease in a human subject of 60 years of age or older.
147. A method of protection against, or prevention of, RSV disease caused by RSV A and/or RSV B in a human subject, for example a human subject of 60 years of age or older, comprising the step of administration to the subject of a composition comprising a trimeric RSV F protein in the prefusion conformation and an adjuvant comprising a saponin and a TLR-4 agonist, wherein the trimeric RSV F protein in the prefusion conformation is derived from RSV A and the composition does not contain an RSV F protein derived from RSV B, wherein the trimeric RSV F protein in the prefusion conformation is present in an amount of 60-180 μg, for example 120 ug and wherein the adjuvant comprises 25 μg of 3D-MPL and 25 μg of QS21, DOPC and cholesterol.
148. A composition comprising a trimeric RSV F protein in the prefusion conformation and an adjuvant comprising a saponin and a TLR-4 agonist for use in protection against or prevention of RSV disease in a human subject 60 years of age or older, wherein the trimeric RSV F protein in the prefusion conformation is derived from RSV A and the composition does not contain an RSV F protein derived from RSV B, wherein the trimeric RSV F protein in the prefusion conformation is present in an amount of 60-180 μg, for example 120 ug and wherein the adjuvant comprises 25 μg of 3D-MPL and 25 μg of QS21, DOPC and cholesterol.

The invention will be further described by reference to the following, non-limiting, figures and examples.

### Example 1—Vaccine Efficacy Against RSV Disease in Adults Aged 60 Years or Older

Example 1 describes the results of a phase 3, randomized, observer-blind, placebo-controlled, multicentre, clinical vaccination trial to demonstrate the prophylactic efficacy and safety, of a candidate RSV vaccine, i.e. RSVPreF3/AS01E vaccine (“RSVPreF3 OA”), when administered intramuscularly to adults aged 60 years and older.

This is an ongoing, phase 3, randomized, observer-blind, placebo-controlled trial (ClinicalTrials.gov: NCT04886596) conducted in 17 countries in Africa, Asia, Australia, Europe and North America. Participants will be followed for three consecutive RSV seasons in the northern hemisphere and at least two consecutive seasons in the southern hemisphere. Below are results after one full season in the northern hemisphere.

The purpose of the study was to demonstrate the efficacy of the RSVPreF3 investigational vaccine in the prevention of RT-PCR-confirmed LRTD caused by RSV A and/or B in adults ≥60 YOA (years of age), following a single dose of the RSVPreF3 OA vaccine and following annual revaccination doses. Experimental design: Phase 3, randomized, observer-blind, placebo-controlled multi-country study with 2 parts:
Part 1 with 4 parallel groups randomized with a ratio of 1:1:1:3 (RSVPreF3 Lot 1/2/3 versus Placebo) before Season 1.
Part 2 with 2 parallel groups randomized with a ratio of 1:1 (RSVPreF3 Lot 4 versus Placebo) before Season 1, which will be initiated when the vaccine lots for Part 1 are no longer available at the study sites.

Each of the 4 RSVPreF3 groups in both parts were randomized before Season 2 into 2 sub-groups (RSV_annual group and RSV_1dose group) with a 1:1 ratio. The RSV_annual group receive an additional dose of RSVPreF3 OA vaccine before each subsequent season, while the RSV_1dose group will receive 1 dose of placebo at the same timepoints. To maintain the study blind, participants who were initially randomized to the Placebo group will also receive additional doses of placebo at the same timepoints. Placebo: saline solution.

TABLE 1
Study objectives and endpoints

| | |
|---|---|
| Objectives | Endpoints |

Primary

| | |
|---|---|
| To demonstrate the efficacy of a single | First occurrence of RT-PCR-confirmed |
| dose of the RSVPreF3 OA investigational | RSV A and/or B-associated LRTD, |
| vaccine in the prevention of RSV-confirmed | according to the case definition*. |
| LRTD during the first season in adults ≥60 | |
| YOA. | |

Secondary
Secondary - Efficacy
Secondary confirmatory

| | |
|---|---|
| To demonstrate the efficacy of a single | First occurrence of RT-PCR-confirmed |
| dose of the RSVPreF3 OA investigational | RSV A and/or B-associated LRTD, |
| vaccine in the prevention of RSV-confirmed | according to the case definition*. |
| LRTD in adults ≥60 YOA over several | |
| seasons. | |
| To demonstrate the efficacy of a single | First occurrence of RT-PCR-confirmed |
| dose of the RSVPreF3 OA investigational | RSV A and/or B-associated LRTD, |
| vaccine followed by annual revaccination | according to the case definition*. |
| doses in the prevention of RSV-confirmed | |
| LRTD in adults ≥60 YOA over several | |
| seasons. | |
| To demonstrate the efficacy of a single | First occurrence of RT-PCR-confirmed |
| dose and annual revaccination doses of | RSV-associated LRTD, according to the |
| the RSVPreF3 OA investigational vaccine | case definition*, for RSV subtype A |
| in the prevention of RSV-confirmed LRTD | and RSV subtype B separately. |
| for each RSV subtype (A and B) separately | |
| in adults ≥60 YOA over 3 seasons. | |

Selected other secondary descriptive

| | |
|---|---|
| To evaluate the efficacy of the RSVPreF3 | First occurrence of RT-PCR-confirmed |
| OA investigational vaccine in the | RSV-associated LRTD, according to the |
| prevention of RSV-confirmed LRTD for each | case definition*, for RSV subtype A |
| RSV subtype (A and B) separately in | and RSV subtype B separately. |
| adults ≥60 YOA, following a single dose | |
| of the RSVPreF3 OA investigational vaccine | |
| and following annual revaccination doses. | |
| To evaluate the efficacy of the RSVPreF3 | First occurrence of RT-PCR-confirmed |
| OA investigational vaccine in the prevention | RSV A and/or B-associated LRTD, |
| of RSV-confirmed LRTD by age category, | according to the case definition*, |
| following a single dose of the RSVPreF3 OA | in the following age categories: |
| investigational vaccine and following annual | ≥65 YOA, ≥70 YOA and ≥80 YOA. |
| revaccination doses. | |
| To evaluate the efficacy of the RSVPreF3 | First occurrence of RT-PCR-confirmed |
| OA investigational vaccine in the prevention | RSV A and/or B-associated LRTD, |
| of RSV-confirmed LRTD by season in | according to the case definition*, |
| adults ≥60 YOA, following a single dose | by season. |
| of the RSVPreF3 OA investigational vaccine | |
| and following annual revaccination doses. | |
| To evaluate the efficacy of the RSVPreF3 | First occurrence of RT-PCR-confirmed |
| OA investigational vaccine in the prevention | RSV A and/or B-associated LRTD, |
| of RSV-confirmed LRTD by year in | according to the case definition*, |
| adults ≥60 YOA, following a single dose of | by year. |
| the RSVPreF3 OA investigational vaccine and | |
| following annual revaccination doses. | |
| To evaluate the evolution of efficacy of a | First occurrence of RT-PCR-confirmed |
| single dose of the RSVPreF3 OA investigational | RSV A and/or B-associated LRTD, |
| vaccine in the prevention of RSV-confirmed | according to the case definition*. |
| LRTD in adults ≥60 YOA over time. | |
| To evaluate the efficacy of the RSVPreF3 | First occurrence of RT-PCR-confirmed |
| OA investigational vaccine in the prevention | RSV A and/or B-associated LRTD according |
| of RSV-confirmed LRTD in adults ≥60 YOA by | to the case definitions*, by baseline |
| baseline comorbidities, following a single | comorbidities. |
| dose of the RSVPreF3 OA investigational | |
| vaccine and following annual revaccination | |
| doses. | |
| To evaluate the efficacy of the RSVPreF3 | First occurrence of RT-PCR-confirmed |
| OA investigational vaccine in the prevention | RSV A and/or B-associated severe LRTD, |
| of severe RSV-confirmed LRTD in | according to the case definitions*. |
| adults ≥60 YOA, following a single dose of | |
| the RSVPreF3 OA investigational vaccine and | |
| following annual revaccination doses. | |
| To evaluate the efficacy of the RSVPreF3 | First occurrence of RT-PCR-confirmed |
| OA investigational vaccine in the prevention | RSV A and/or B-associated ARI, according |
| of RSV-confirmed ARI in adults ≥60 YOA, | to the case definition*. |
| following a single dose of the RSVPreF3 OA | |
| investigational vaccine and following | |
| annual revaccination doses. | |

Secondary - Immunogenicity

| | |
|---|---|
| To evaluate the humoral immune response to | In a subset of participants, at pre- |
| the RSVPreF3 OA investigational vaccine. | Dose 1 (Day 1), 30 days post-Dose 1 |
| | (Day 31), pre-Dose 2 (pre-Season 2) |
| | and pre-Dose 3 (pre-Season 3): |
| | RSVPreF3 IgG-specific antibody concentrations. |
| | Neutralizing antibody titers against RSV A. |
| | Neutralizing antibody titers against RSV B. |

Secondary - Safety

| | |
|---|---|
| To evaluate the reactogenicity of the | In a subset of participants, occurrence, |
| RSVPreF3 OA investigational vaccine. | intensity and duration of solicited |
| | administration site and systemic events |
| | with an onset during the 4-day follow-up |
| | period after each vaccination (i.e., the |
| | day of vaccination and 3 subsequent days). |
| To evaluate the safety of the RSVPreF3 | In all participants: |
| OA investigational vaccine. | Occurrence of unsolicited AEs with an |
| | onset during the 30-day follow-up period |
| | after each vaccination (i.e., the day of |
| | vaccination and 29 subsequent days). |
| | Occurrence of all serious adverse events |
| | (SAEs) from the day of vaccination up to 6 |
| | months after each-vaccination. |
| | Occurrence of all pIMDs from the day of |
| | vaccination up to 6 months after each |
| | vaccination. |
| | Occurrence of SAEs related to study vaccination |
| | from Day 1 up to study end. |
| | Occurrence of pIMDs related to study vaccination |
| | from Day 1 up to study end. |
| | Occurrence of any fatal SAEs from Day 1 up to |
| | study end. |

### Population(s) Recruited:

Persons ≥60-year-olds not previously or currently enrolled in another RSV vaccine trial were enrolled. Persons with chronic medical conditions were eligible if they were considered medically stable by the investigator. In total, 12,467 participants received RSVPreF3 OA and 12,499 received placebo. Of these, 39.6% (RSVPreF3 OA) and 38.9% (placebo) had ≥1 pre-existing medical condition of interest. Before the RSV season, participants were randomized (1:1) to receive RSVPreF3 OA or placebo, using an automated internet-based system. Each 0.5 mL dose of reconstituted RSVPreF3 OA contained 120 μg RSVPreF3 antigen and the liposome-based AS01E Adjuvant System containing 25 μg 3-O-desacyl-4′-monophosphoryl lipid A and 25 μg QS-21 (Quillaja saponaria Molina, fraction 21). RSVPreF3 OA or placebo (saline) was injected in the deltoid muscle of the non-dominant arm. Injections were administered by personnel not involved in data collection/evaluation. Participants and study team members responsible for evaluating endpoints were blinded.

TABLE 2

| | RSVPreF3 | | Placebo | | Total | |
|---|---|---|---|---|---|---|
| | N = 12467 | | N = 12499 | | N = 24966 | |
| | Value or n | % | Value or n | % | Value or n | % |
| Age (years) at vaccination at Visit 1 | | | | | | |
| n | 12467 | | 12499 | | 24966 | |
| Mean | 69.5 | | 69.6 | | 69.5 | |
| Age category | | | | | | |
| >=65 YOA | 9259 | 74.3 | 9329 | 74.6 | 18588 | 74.5 |
| >=70 YOA | 5504 | 44.1 | 5519 | 44.2 | 11023 | 44.2 |
| >=80 YOA | 1017 | 8.2 | 1028 | 8.2 | 2045 | 8.2 |
| 60-69 YOA | 6963 | 55.9 | 6980 | 55.8 | 13943 | 55.8 |
| 70-79 YOA | 4487 | 36.0 | 4491 | 35.9 | 8978 | 36.0 |
| Sex | | | | | | |
| Male | 5979 | 48.0 | 6072 | 48.6 | 12051 | 48.3 |
| Female | 6488 | 52.0 | 6427 | 51.4 | 12915 | 51.7 |
| Race | | | | | | |
| American Indian Or Alaska Native | 44 | 0.4 | 35 | 0.3 | 79 | 0.3 |
| Asian | 953 | 7.6 | 956 | 7.6 | 1909 | 7.6 |
| Black Or African American | 1064 | 8.5 | 1101 | 8.8 | 2165 | 8.7 |
| Native Hawaiian Or Other Pacific Islander | 11 | 0.1 | 6 | 0.0 | 17 | 0.1 |
| White | 9887 | 79.3 | 9932 | 79.5 | 19819 | 79.4 |
| Other | 508 | 4.1 | 469 | 3.8 | 977 | 3.9 |
| Hemisphere | | | | | | |
| Northern hemisphere | 11496 | 92.2 | 11522 | 92.2 | 23018 | 92.2 |
| Southern hemisphere | 971 | 7.8 | 977 | 7.8 | 1948 | 7.8 |

### Selection of Pre-Existing Medical Conditions of Interest (Comorbidities)

Subgroup analyses reported here were based on the presence or absence of selected pre-existing medical conditions that are known to increase the risk of severe RSV disease [Centers for Disease Control and Prevention. RSV in older adults and adults with chronic medical conditions. Available at: https://www.cdc.gov/rsv/high-risk/older-adults.html], complemented with relevant risk factors of influenza complications [Centers for Disease Control and Prevention. People at higher risk of flu complications. Available at: https://www.cdc.gov/flu/highrisk/index.htm], collectively referred to as “conditions of interest”. These included cardiorespiratory conditions (COPD, asthma, any chronic respiratory or pulmonary disease, and chronic heart failure) and endocrine and metabolic conditions (diabetes mellitus type 1 or 2 and advanced liver or renal disease). Information about pre-existing medical conditions was gathered by interviewing the participants and/or reviewing their medical records.

### Pre-Existing Medical Conditions (Comorbidities)

More than 95.0% of participants in both groups had at least one pre-existing general medical condition. The most common conditions were vascular hypertensive disorders (57.2% of all participants in the exposed population), osteoarthropathies (33.5%), elevated cholesterol (24.8%), and diabetes mellitus (22.9%). In total, 39.6% (RSVPreF3 OA) and 38.9% (placebo) of participants had at least one of the pre-existing medical conditions of interest (i.e., associated with severe RSV disease); 20.0% and 19.4% had at least one cardiorespiratory condition of interest, and 25.7% and 25.9% had at least one endocrine or metabolic condition of interest (mostly diabetes). The mean body mass index in both groups was 29.1 kg/m2. Other baseline characteristics were also balanced between groups.

TABLE 3

| | RSVPreF3 | | Placebo | | Total | |
|---|---|---|---|---|---|---|
| | N = 12467 | | N = 12499 | | N = 24966 | |
| | Value or n | % | Value or n | % | Value or n | % |
| Charlson Comorbidity Index - Categories | | | | | | |
| Low/Medium risk | 8235 | 66.1 | 8368 | 66.9 | 16603 | 66.5 |
| High risk | 4232 | 33.9 | 4131 | 33.1 | 8363 | 33.5 |
| Comorbidity of interest | | | | | | |
| At least 1 pre-existing comorbidity of interest | 4937 | 39.6 | 4864 | 38.9 | 9801 | 39.3 |
| At least 1 pre-existing Cardiorespiratory condition | 2496 | 20.0 | 2422 | 19.4 | 4918 | 19.7 |
| At least 1 pre-existing Endocrinometabolic condition | 3200 | 25.7 | 3236 | 25.9 | 6436 | 25.8 |

### Analysis Sets:

TABLE 4

| Population | RSVPreF3 | Placebo | Total | Comment |
|---|---|---|---|---|
| Exposed set | 12467 | 12499 | 24966 | N = 24981 vaccinated, |
| (ES) | | | | 15 participants excluded |
| | | | | due to invalid ICF |
| | | | | (code 900) |
| Modified ES | 12466 | 12494 | 24960 | 6 participants reported RSV |
| (mES) | | | | ARI <15 days post- |
| | | | | vaccination and are |
| | | | | excluded |
| Solicited | 879 | 878 | 1757 | 42 participants did not |
| safety set | | | | report solicited event |
| | | | | information |

### Efficacy Assessments:

ARI surveillance was done via spontaneous reporting by participants and actively through scheduled contacts between site staff and participants. Starting from the day of vaccination, participants had to contact the site staff after experiencing at least two ARI symptoms/signs for 24 hours (Table 5). Starting from 30 days post-dose 1, the site staff contacted participants every 2 weeks during the RSV season and every month during interseasons to capture ARI not spontaneously reported by participants. Participants had to take a nasal self-swab (preferably within 48 hours after symptom onset) and contact the site to plan an ARI visit, during which additional nasal and throat swabs were collected by study personnel for confirmed ARIs. Swabs were tested for RSV-A/RSV-B by quantitative reverse transcription-polymerase chain reaction (qRT-PCR). Each ARI episode was followed up with additional contacts until resolution.

ARI was defined by at least two respiratory symptoms/signs or at least one respiratory and one systemic symptom/sign for ≥24 hours. LRTD was defined by at least two lower respiratory symptoms/signs, including at least one lower respiratory sign or at least three lower respiratory symptoms for ≥24 hours (Table 5). An external adjudication committee reviewed all RSV-LRTD cases fulfilling the case definition and all investigator-reported RSV-LRTD cases. The primary efficacy analysis included externally adjudicated cases only.

TABLE 5
Case definitions for evaluation of vaccine efficacy

| Endpoint | Case definition | |
|---|---|---|
| ARI | Presence of: | |
| (Trigger for swabbing) | at least 2 respiratory symptoms/signs for at least 24 hours | |
| | OR | |
| | at least 1 respiratory symptom/sign + 1 systemic symptom/sign for at least 24 hours | |
| | Respiratory symptoms and signs | Systemic symptoms and signs |
| | Nasal congestion/rhinorrhea | Fever¹/feverishness² |
| | Sore throat | Fatigue |
| | New or increased sputum | Body aches |
| | New or increased cough | Headache |
| | New or increased dyspnea | Decreased appetite |
| | (shortness of breath) | |
| | New or increased wheezing³ | |
| | New or increased crackles/ronchi⁴ | |
| | based on chest auscultation | |
| | Respiratory rate ≥20 respirations/min⁴ | |
| | Low or decreased oxygen saturation | |
| | (=O₂ saturation <95% or ≤90% | |
| | if pre-season baseline is <95%)⁴ | |
| | Need for oxygen supplementation⁴ | |
| RT-PCR-confirmed RSV-ARI ⁵ | An event meeting the case definition of ARI with at least | |
| | one RSV-positive swab detected by RT-PCR.^{Error! Reference source not found.} | |
| LRTD | Presence of: | |
| | at least 2 lower respiratory symptoms/signs for at least 24 | |
| | hours including at least 1 lower respiratory SIGN | |
| | OR | |
| | at least 3 lower respiratory symptoms for at least 24 hours | |
| | Lower respiratory symptoms | Lower respiratory signs |
| | New or increased sputum | New or increased wheezing³ |
| | New or increased cough | New or increased crackles/ronchi⁴ |
| | | based on chest auscultation |
| | New or increased dyspnea | Respiratory rate ≥20 respirations/min⁴ |
| | (shortness of breath) | Low or decreased oxygen saturation |
| | | (=O₂ saturation <95% or ≤90% |
| | | if pre-season baseline is <95%)⁴ |
| | | Need for oxygen supplementation⁴ |

| RT-PCR-confirmed RSV-LRTD⁵ | An event meeting the case definition of LRTD with at least one |
|---|---|
| | RSV-positive swab detected by RT-PCR. |
| RT-PCR-confirmed severe | Presence of a LRTD with at least one of the following criteria: |
| RSV LRTD - Definition 1 | at least 2 lower respiratory SIGNS |
| “Clinical symptomology” ⁵ | an LRTD episode assessed as ‘severe’ by the investigator^{Error! Reference source not found.} |
| | AND |
| | with at least one RSV-positive detected by RT-PCR |
| | Lower respiratory signs |
| | New or increased wheezing³ |
| | New or increased crackles/ronchi⁴ based on chest auscultation |
| | Respiratory rate ≥20 respirations/min⁴ |
| | Low or decreased oxygen saturation (=O₂ saturation <95% or ≤90% |
| | if pre-season baseline is <95%)⁴ |
| | Need for oxygen supplementation⁴ |
| RT-PCR-confirmed severe | Presence of a LRTD with at least one of the following criteria^{Error! Reference source not found.}: |
| RSV LRTD-Definition 2 | Need for oxygen supplementation⁴ |
| “Supportive therapy“ ⁵ | Need for positive airway pressure therapy (e.g. CPAP) |
| | Need for other types of mechanical ventilation |
| | AND |
| | with at least one RSV-positive swab detected by RT-PCR |

ARI: acute respiratory infection;
LRTD: lower respiratory tract disease;
RSV: respiratory syncytial virus
¹Fever is defined as a temperature ≥38.0° C./100.4° F. by any route.
²Feverishness is defined as the feeling of having fever without objective measurement.
³Reported by study participant or investigator.
⁴Reported by investigator.
⁵Throat and/or nasal swab samples collected at ARI visits for RT-PCR testing will be collected within 6 days after ARI onset (i.e., up to Day 7). In special circumstances (for example in case of suspected COVID-19 infection and pending COVID-19 test result, or self-quarantine) and if it is not possible to perform the ARI visit within 6 days after ARI onset (i.e., within Day 3 to Day 7), then the interval for this visit and the site swab collection may be extended up to maximum 14 days after ARI onset (i.e., until Day 15).

### Results at Analysis 1 Time Point:

Primary objective—RSV LRTD (mES)—Vaccine efficacy against first occurrence of RT-PCR-confirmed RSV LRTD up to vaccine efficacy analysis 1 using Poisson method (mES)

TABLE 6

| | RSVPreF3 | | | | Placebo | | | | VE | | | |
|---|---|---|---|---|---|---|---|---|---|---|---|---|
| | | | | n/T (per | | | | n/T (per | | 96.95% CI | | P- |
| Endpoint | N | n | T(year) | 1000) | N | n | T(year) | 1000) | % | LL | UL | value |
| RT-PCR- | 12466 | 7 | 6865.9 | 1.0 | 12494 | 40 | 6857.3 | 5.8 | 82.58 | 57.89 | 94.08 | <0.0001 |
| confirmed RSV | | | | | | | | | | | | |
| LRTD | | | | | | | | | | | | |

Secondary objective - RSV LRTD by Age Category (mES) - Vaccine efficacy against first occurrence of RT-PCR-confirmed RSV LRTD up to vaccine efficacy analysis 1 using Poisson method (mES)

TABLE 7

| | RSVPreF3 | | | | Placebo | | | | VE | | | |
|---|---|---|---|---|---|---|---|---|---|---|---|---|
| | | | | n/T (per | | | | n/T (per | | 95% CI | | P- |
| Endpoint | N | n | T(year) | 1000) | N | n | T(year) | 1000) | % | LL | UL | value |
| By subtype | | | | | | | | | | | | |
| RSV-A LRTD | 12466 | xx | 6867.4 | xx | 12494 | xx | 6868.9 | xx | 84.62 | 32.08 | 98.32 | 0.0074 |
| RSV-B LRTD | 12466 | xx | 6866.7 | xx | 12494 | xx | 6862.3 | xx | 80.88 | 49.40 | 94.27 | 0.0002 |
| By Age category | | | | | | | | | | | | |
| >=65 YOA | 9258 | 5 | 5098.7 | 1.0 | 9325 | 29 | 5132.1 | 5.7 | 82.72 | 54.85 | 94.78 | <0.0001 |
| >=70 YOA | 5503 | 3 | 3015.0 | 1.0 | 5515 | 19 | 3020.9 | 6.3 | 84.37 | 46.91 | 97.04 | 0.0008 |
| >=80 YOA | 1016 | 2 | 551.4 | 3.6 | 1028 | 3 | 559.3 | 5.4 | 33.83 | −477.68 | 94.47 | 0.9931 |
| 60-69 YOA | 6963 | 4 | 3850.8 | 1.0 | 6979 | 21 | 3836.4 | 5.5 | 80.96 | 43.56 | 95.25 | 0.0009 |
| 70-79 YOA | 4487 | 1 | 2463.6 | 0.4 | 4487 | 16 | 2461.6 | 6.5 | 93.81 | 60.15 | 99.85 | 0.0003 |
| By baseline | | | | | | | | | | | | |
| comorbidities | | | | | | | | | | | | |
| Low/Medium risk | 8235 | 4 | 4495.8 | 0.9 | 8367 | 23 | 4560.6 | 5.0 | 82.39 | 48.45 | 95.57 | 0.0004 |
| (Charlson index) | | | | | | | | | | | | |
| High risk (Charlson | 4231 | 3 | 2370.0 | 1.3 | 4127 | 17 | 2296.6 | 7.4 | 82.88 | 40.79 | 96.79 | 0.0021 |
| index) | | | | | | | | | | | | |
| No pre-existing | 7529 | 6 | 4094.1 | 1.5 | 7633 | 22 | 4148.1 | 5.3 | 72.46 | 29.97 | 90.87 | 0.0040 |
| comorbidity of interest | | | | | | | | | | | | |
| At least 1 pre-existing | 4937 | 1 | 2771.8 | 0.4 | 4861 | 18 | 2709.1 | 6.6 | 94.61 | 65.88 | 99.87 | <0.0001 |
| comorbidity of interest | | | | | | | | | | | | |
| At least 1 pre-existing | xx | xx | xx | xx | xx | xx | xx | xx | xx | xx | xx | xx |
| Cardiorespiratory condition | | | | | | | | | | | | |
| At least 1 pre-existing | xx | xx | xx | xx | xx | xx | xx | xx | xx | xx | xx | xx |
| Endocrinometabolic | | | | | | | | | | | | |
| condition | | | | | | | | | | | | |

Vaccine Efficacy Against RSV Disease in Participants with Pre-Existing Medical Conditions:

The RSV-LRTD incidence rates in the placebo group tended to be higher among participants with the selected pre-existing conditions of interest (6.6-8.9/1000 person-years) than among those without any of these conditions (5.3/1000 person-years). A similar observation was made for the RSV-ARI incidence rates, which were 15.2-17.8/1000 person-years among placebo recipients with pre-existing conditions of interest and 13.1/1000 person-years among those without these conditions (Table 8). Efficacy of RSVPreF3 OA against RSV-LRTD was 94.6% (95% CI, 65.9-99.9) in participants with at least one of the conditions of interest (92.1% [46.7-99.8] among those with cardiorespiratory conditions and 100% [74.0-100] among those with endocrine or metabolic conditions) and 92.0% (46.1-99.8) in participants with at least two of these conditions (Table 8).

Efficacy of RSVPreF3 OA against RSV-ARI was 81.0% (95% CI, 58.9-92.3) in participants with at least one of the medical conditions of interest (88.1% [60.9-97.7] among those with cardiorespiratory conditions and 79.4% [49.4-93.0] among those with endocrine or metabolic conditions) and 88.0% (60.5-97.7) in participants with at least two of these conditions (Table 8).

TABLE 8

| | RSVPreF3 OA | | | | Placebo | | | | Vaccine | |
|---|---|---|---|---|---|---|---|---|---|---|
| | | | T, | n/T, | | | T, | n/T, | efficacy, | |
| Endpoint | N | n | p-yr | n/1000 p-yr | N | n | p-yr | n/1000 p-yr | % (CI^{a}) | |
| RSV-LRTD | | | | | | | | | | |
| RSV-LRTD, overall | 12,466 | 7 | 6865.9 | 1.0 | 12,494 | 40 | 6857.3 | 5.8 | 82.6 | (57.9-94.1) |
| RSV-LRTD with medically attended visits^{b}, overall | 12,466 | 3 | 6867.1 | 0.4 | 12,494 | 24 | 6864.0 | 3.5 | 87.5 | (58.9-97.6) |
| RSV-LRTD by co-existing condition of interest^{c} | | | | | | | | | | |
| No condition of interest | 7529 | 6 | 4094.1 | 1.5 | 7633 | 22 | 4148.1 | 5.3 | 72.5 | (30.0-90.9) |
| ≥1 condition of interest | 4937 | 1 | 2771.8 | 0.4 | 4861 | 18 | 2709.1 | 6.6 | 94.6 | (65.9-99.9) |
| ≥1 cardiorespiratory condition of interest^{d} | 2496 | 1 | 1409.5 | 0.7 | 2421 | 12 | 1352.9 | 8.9 | 92.1 | (46.7-99.8) |
| ≥1 endocrine and metabolic condition of interest^{e} | 3200 | 0 | 1795.7 | 0.0 | 3234 | 13 | 1805.3 | 7.2 | 100 | (74.0-100) |
| ≥2 conditions of interest | 2504 | 1 | 1418.2 | 0.7 | 2431 | 12 | 1362.8 | 8.8 | 92.0 | (46.1-99.8) |
| RSV-ARI | | | | | | | | | | |
| RSV-ARI, overall | 12,466 | 27 | 6858.7 | 3.9 | 12,494 | 95 | 6837.8 | 13.9 | 71.7 | (56.2-82.3) |
| RSV-ARI with medically attended visits^{b}, overall | 12,466 | 8 | 6865.9 | 1.2 | 12,494 | 38 | 6859.9 | 5.5 | 79.0 | (54.3-91.5) |
| RSV-ARI by co-existing condition of interest^{c} | | | | | | | | | | |
| No condition of interest | 7529 | 19 | 4089.9 | 4.6 | 7633 | 54 | 4136.4 | 13.1 | 64.4 | (39.0-80.1) |
| ≥1 condition of interest | 4937 | 8 | 2768.8 | 2.9 | 4861 | 41 | 2701.4 | 15.2 | 81.0 | (58.9-92.3) |
| ≥1 cardiorespiratory condition of interest^{d} | 2496 | 3 | 1408.5 | 2.1 | 2421 | 24 | 1349.0 | 17.8 | 88.1 | (60.9-97.7) |
| ≥1 endocrine and metabolic condition of interest^{e} | 3200 | 6 | 1793.2 | 3.3 | 3234 | 29 | 1800.0 | 16.1 | 79.4 | (49.4-93.0) |
| ≥2 conditions of interest | 2504 | 3 | 1417.3 | 2.1 | 2431 | 24 | 1358.8 | 17.7 | 88.0 | (60.5-97.7) |

Cases were confirmed as positive for RSV-A or RSV-B subtypes by quantitative reverse transcriptase-polymerase chain reaction. RSV-LRTD cases were those identified by the adjudication committee. Vaccine efficacy was estimated using the Poisson method, with adjustment for age and geographic region. RSVPreF3 OA, group with participants who received a single dose of AS01_{E}-adjuvanted RSV prefusion F protein-based candidate vaccine; placebo, group with participants who received a single dose of placebo; N, number of participants in the modified exposed population in the specified subgroup; n, number of participants with at least one RSV-LRTD or RSV-ARI; T, sum of follow-up time (from day 15 post-vaccination until first occurrence of the event, data lock point, or drop-out); p-yr, person-years; n/T, incidence rate of participants reporting at least one event; CI, confidence interval.
^{a}96.95% CI for primary endpoint (RSV-LRTD, overall); 95% CI for other endpoints (no adjustment for multiplicity).
^{b}Medically attended visits included visits with a general practitioner or specialist, emergency department visits, intensive care unit admissions, and hospitalizations.
^{c}Co-existing medical conditions of interest included cardiorespiratory conditions (chronic obstructive pulmonary disease, asthma, any chronic respiratory or pulmonary disease, chronic heart failure) and endocrine and metabolic conditions (diabetes mellitus type 1 or type 2 and advanced liver or renal disease) that are associated with an increased risk of severe RSV disease.
^{d}Of the 13 RSV-LRTD and 27 RSV-ARI cases among participants with cardiorespiratory conditions of interest, 13 RSV-LRTD and 24 RSV-ARI cases were among participants with chronic respiratory or pulmonary disease.
^{e}Of the 13 RSV-LRTD and 35 RSV-ARI cases among participants with endocrine and metabolic conditions of interest, 12 RSV-LRTD and 34 RSV-ARI cases were among participants with diabetes mellitus.
Secondary objective - severe RSV LRTD - Vaccine efficacy against first occurrence of RT-PCR-confirmed RSV severe LRTD up to vaccine efficacy analysis 1 using Poisson method (mES)

TABLE 9

| | | RSVPreF3 | | | | Placebo | | | | | | | |
|---|---|---|---|---|---|---|---|---|---|---|---|---|---|
| | | | | | n/T | | | | n/T | VE | | | |
| | | | | | (per | | | | (per | | 95% CI | | P- |
| Endpoint | Definition | N | n | T(year) | 1000) | N | n | T(year) | 1000) | % | LL | UL | value |
| RT-PCR-confirmed RSV | Any | 12466 | 1 | 6867.9 | 0.1 | 12494 | 17 | 6867.7 | 2.5 | 94.10 | 62.37 | 99.86 | 0.0001 |
| severe LRTD | | | | | | | | | | | | | |
| | Definition 1: | xx | xx | xx | xx | xx | xx | xx | xx | xx | xx | xx | xx |
| | Clinical | | | | | | | | | | | | |
| | symptomology | | | | | | | | | | | | |
| | Definition 2: | xx | xx | xx | xx | xx | xx | xx | xx | xx | xx | xx | xx |
| | Supportive | | | | | | | | | | | | |
| | therapy | | | | | | | | | | | | |

Secondary objective - RSV ARI - Vaccine efficacy against first occurrence of RT-PCR-confirmed RSV ARI up to vaccine efficacy analysis 1 - any, and by RSV sub-type using Poisson method (mES)

TABLE 10

| | RSVPreF3 | | | | Placebo | | | | | | | |
|---|---|---|---|---|---|---|---|---|---|---|---|---|
| | | | | n/T | | | | n/T | VE | | | |
| | | | | (per | | | | (per | | 95% CI | | P- |
| | N | n | T(year) | 1000) | N | n | T(year) | 1000) | % | LL | UL | value |
| Endpoint | | | | | | | | | | | | |
| RT-PCR confirmed RSV ARI | 12466 | 27 | 6858.7 | 3.9 | 12494 | 95 | 6837.8 | 13.9 | 71.71 | 56.23 | 82.27 | <0.0001 |
| By subtype | | | | | | | | | | | | |
| RSV-A ARI | 12466 | xx | 6865.2 | xx | 12494 | xx | 6862.3 | xx | 71.89 | 39.74 | 88.20 | 0.0004 |
| RSV-B ARI | 12466 | xx | 6861.7 | xx | 12494 | xx | 6849.4 | xx | 70.62 | 49.62 | 83.66 | <0.0001 |

TABLE 10
Summary of Vaccine Efficacy Results:

| Objective | Endpoint | | Categories | VE | LL | UL |
|---|---|---|---|---|---|---|
| Primary | RSV LRTD | All | | 82.58 | 57.89 | 94.08 |
| Secondary | RSV LRTD | By subtype | RSV A | 84.62 | 32.08 | 98.32 |
| | | | RSV B | 80.88 | 49.40 | 94.27 |
| | | by Age | >=65 | 82.72 | 54.85 | 94.78 |
| | | | >=70 | 84.37 | 46.91 | 97.04 |
| | | | >=80 | 33.83 | −477.68 | 194.47 |
| | | | 60-69 | 80.96 | 43.56 | 95.25 |
| | | | 70-79 | 93.81 | 60.15 | 99.85 |
| | | Severe | | 94.10 | 62.37 | 99.86 |
| | | By | No pre-existing | 72.46 | 29.97 | 90.87 |
| | | comorbidities | comorbidity | | | |
| | | | of interest | | | |
| | | | At least 1 pre-existing | 94.61 | 65.88 | 99.87 |
| | | | comorbidity of interest | | | |
| | RSV ARI | All | | 71.71 | 56.23 | 82.27 |
| | | By subtype | RSV A | 71.89 | 39.74 | 88.20 |
| | | | RSV B | 70.62 | 49.62 | 83.66 |

Primary confirmatory objective is met with LL (96.95% CIs) of 57.89%. Very high estimated VE of 82.58%
Secondary descriptive objectives (95% CIs): consistently high efficacy observed for all secondary efficacy endpoints, except vaccine efficacy in 80+ yoa participants which is inconclusive owing to smaller sample size

### Safety Assessments:

Participants in the reactogenicity/immunogenicity subset used paper diaries to record solicited injection site and systemic reactions starting within 4 days post-vaccination. Unsolicited adverse events (AEs) starting within 30 days post-vaccination were recorded by all participants (exposed set, ES) in paper diaries. Participants not part of the reactogenicity/immunogenicity subset recorded all AEs within 30 days as unsolicited AEs, including reactogenicity events. AE intensity was graded from mild (grade 1) to severe (grade 3) (by the participants for solicited, by the investigators for unsolicited AEs). Serious AEs (SAEs) and potential immune-mediated diseases were collected from the day of vaccination until 6 months post-vaccination. SAEs and potential immune-mediated diseases considered related to vaccination by investigator assessment, fatal SAEs and(S) AEs leading to withdrawal are recorded until study end.

### Safety Results:

Secondary objective—reactogenicity events—review does not raise any safety concern—profile clinically acceptable and in line with expectations for adjuvanted vaccines

TABLE 11

| | | RSVPreF3 | | | | Placebo | | | |
|---|---|---|---|---|---|---|---|---|---|
| | | | | 95% CI | | | | 95% CI | |
| | | n | % | LL | UL | n | % | LL | UL |
| Erythema (mm) | | | | | | | | | |
| Vaccination at visit 1 | N | 879 | | | | 874 | | | |
| | Any | 66 | 7.5 | 5.9 | 9.5 | 7 | 0.8 | 0.3 | 1.6 |
| | >20 and <=50 | xx | xx | xx | xx | xx | xx | xx | xx |
| | >50 and <=100 | xx | xx | xx | xx | xx | xx | xx | xx |
| | >100 | xx | xx | xx | xx | xx | xx | xx | xx |
| | Medically attended | 0 | 0 | 0 | 0.4 | 0 | 0 | 0 | 0.4 |
| | visits | | | | | | | | |
| Swelling (mm) | | | | | | | | | |
| Vaccination at visit 1 | N | 879 | | | | 874 | | | |
| | Any | 48 | 5.5 | 4.1 | 7.2 | 5 | 0.6 | 0.2 | 1.3 |
| | >20 and <=50 | xx | xx | xx | xx | xx | xx | xx | xx |
| | >50 and <=100 | xx | xx | xx | xx | xx | xx | xx | xx |
| | >100 | xx | xx | xx | xx | xx | xx | xx | xx |
| | Medically attended | 0 | 0 | 0 | 0.4 | 0 | 0 | 0 | 0.4 |
| | visits | | | | | | | | |
| Pain | | | | | | | | | |
| Vaccination at visit 1 | N | 879 | | | | 874 | | | |
| | Any | 535 | 60.9 | 57.5 | 64.1 | 81 | 9.3 | 7.4 | 11.4 |
| | Grade 1 | xx | xx | xx | xx | xx | xx | xx | xx |
| | Grade 2 | xx | xx | xx | xx | xx | xx | xx | xx |
| | Grade 3 | xx | xx | xx | xx | xx | xx | xx | xx |
| | Medically attended | 0 | 0 | 0 | 0.4 | 0 | 0 | 0 | 0.4 |
| | visits | | | | | | | | |
| Arthralgia | | | | | | | | | |
| Vaccination at visit 1 | N | 879 | | | | 878 | | | |
| | Any | 159 | 18.1 | 15.6 | 20.8 | 56 | 6.4 | 4.9 | 8.2 |
| | Grade 1 | 102 | 11.6 | 9.6 | 13.9 | 37 | 4.2 | 3.0 | 5.8 |
| | Grade 2 | 46 | 5.2 | 3.9 | 6.9 | 13 | 1.5 | 0.8 | 2.5 |
| | Grade 3 | 11 | 1.3 | 0.6 | 2.2 | 5 | 0.6 | 0.2 | 1.3 |
| | Medically attended | 0 | 0 | 0 | 0.4 | 0 | 0 | 0 | 0.4 |
| | visits | | | | | | | | |
| Fatigue | | | | | | | | | |
| Vaccination at visit 1 | N | 879 | | | | 878 | | | |
| | Any | 295 | 33.6 | 30.4 | 36.8 | 141 | 16.1 | 13.7 | 18.7 |
| | Grade 1 | 205 | 23.3 | 20.6 | 26.3 | 113 | 12.9 | 10.7 | 15.3 |
| | Grade 2 | 75 | 8.5 | 6.8 | 10.6 | 23 | 2.6 | 1.7 | 3.9 |
| | Grade 3 | 15 | 1.7 | 1.0 | 2.8 | 4 | 0.5 | 0.1 | 1.2 |
| | Medically attended | *1* | | | | *1* | | | |
| | visits | | | | | | | | |
| Headache | | | | | | | | | |
| Vaccination at visit 1 | N | 879 | | | | 878 | | | |
| | Any | 239 | 27.2 | 24.3 | 30.3 | 111 | 12.6 | 10.5 | 15.0 |
| | Grade 1 | xx | xx | xx | xx | xx | xx | xx | xx |
| | Grade 2 | xx | xx | xx | xx | xx | xx | xx | xx |
| | Grade 3 | xx | xx | xx | xx | xx | xx | xx | xx |
| | Medically attended | 0 | 0 | 0 | 0.4 | 0 | 0 | 0 | 0.4 |
| | visits | | | | | | | | |
| Myalgia | | | | | | | | | |
| Vaccination at visit 1 | N | 879 | | | | 878 | | | |
| | Any | 254 | 28.9 | 25.9 | 32.0 | 72 | 8.2 | 6.5 | 10.2 |
| | Grade 1 | 181 | 20.6 | 18.0 | 23.4 | 56 | 6.4 | 4.9 | 8.2 |
| | Grade 2 | 61 | 6.9 | 5.3 | 8.8 | 12 | 1.4 | 0.7 | 2.4 |
| | Grade 3 | 12 | 1.4 | 0.7 | 2.4 | 3 | 0.3 | 0.1 | 1.0 |
| | Medically attended | *1* | | | | *1* | | | |
| | visits | | | | | | | | |
| Fever (° C.) | | | | | | | | | |
| Vaccination at visit 1 | N | 879 | | | | 878 | | | |
| | >=38-<=38.5 | xx | xx | xx | xx | xx | xx | xx | xx |
| | >38.5-<=39 | xx | xx | xx | xx | xx | xx | xx | xx |
| | >=38.0 | 18 | 2.0 | 1.2 | 3.2 | 3 | 0.3 | 0.1 | 1.0 |
| | >38.5 | 6 | 0.7 | 0.3 | 1.5 | 3 | 0.3 | 0.1 | 1.0 |
| | >39.0 | 1 | 0.1 | 0.0 | 0.6 | 1 | 0.1 | 0.0 | 0.6 |
| | >39.5 | *1* | | | | *1* | | | |
| | >40.0 | 0 | 0 | 0 | 0.4 | 0 | 0 | 0 | 0.4 |
| | Medically attended | 0 | 0 | 0 | 0.4 | 0 | 0 | 0 | 0.4 |
| | visits | | | | | | | | |

### Statistical Analyses

The primary efficacy analysis was performed on the modified exposed set (mES, participants in the ES who did not report an RSV-ARI before day 15 post-vaccination). Additional analyses were performed on the ES and the per-protocol set for efficacy (PPSe, protocol-compliant participants in the mES with efficacy data available). The primary objective was demonstrated if the lower limit (LL) of the two-sided confidence interval (CI) around the efficacy estimate was >20%. The current primary efficacy analysis was performed (as planned), as ≥35 RSV-LRTD had been accrued in the primary cohort for efficacy based on data available at the end of the first northern-hemisphere RSV season. The type I error was adjusted to maintain the overall significance level, and a two-sided 96.95% CI was calculated for the primary objective. Vaccine efficacy was calculated as 1 minus the relative risk using the conditional exact binomial method based on the Poisson model. Periods at risk ended at first event occurrence or censoring and started on day 15 post-vaccination for mES and PPSe analyses and on the day of vaccination for ES analyses. Vaccine efficacy was assessed in participants who (at baseline) reported none of the medical conditions of interest, at least one of these conditions, at least one of the cardiorespiratory, or at least one of the endocrine and metabolic conditions of interest. In addition, efficacy was analyzed post-hoc in participants with at least two conditions of interest.

As the primary endpoint was demonstrated in the current analysis, the results are considered final for the primary objective.

Safety endpoints were analyzed on the ES, except for solicited reactions, which were analyzed on the solicited safety set (SSS, participants in the reactogenicity/immunogenicity subset with solicited safety data available).

Immunogenicity was analyzed on the per-protocol set for immunogenicity (PPSi, protocol-compliant participants in the reactogenicity/immunogenicity subset with immunogenicity data available). All statistical analyses were performed using SAS Life Science Analytics Framework.

### Summary of Results

24,966 participants received one dose of RSVPreF3 OA (n=12,467) or placebo (n=12,499). VE against RSV-LRTD was 82.6% (96.95% CI, 57.9-94.1), with 7 RSV-LRTD cases in vaccine and 40 in placebo recipients during a median follow-up of 6.7 months. VE was 94.1% (95% CI, 62.4-99.9) against severe RSV-LRTD and 71.7% (56.2-82.3) against RSV-ARI. RSV-A- and RSV-B-subtype-specific VE was comparable (84.6% and 80.9% for RSV-LRTD; 71.9% and 70.6% for RSV-ARI).

High VE was observed in different age groups and in participants with comorbidities. RSVPreF3 OA was more reactogenic than placebo, but most solicited adverse events were transient, with mild-to-moderate severity. Rates of serious adverse events and potential immune-mediated diseases were similar in both study groups.

### CONCLUSIONS

A single RSVPreF3 OA dose was highly efficacious in preventing RSV-LRTD and RSV-ARI in ≥60-year-olds, regardless of respiratory disease severity, RSV subtype and underlying comorbidities. No safety concerns were identified.

1. A method of protection against, or prevention of RSV disease in a human subject of 70 years of age or older, comprising the step of administering to the subject a composition comprising (i) a trimeric RSV F protein comprising the amino acid sequence of SEQ ID NO:2; and (ii) an adjuvant comprising QS21, cholesterol, 3D-MPL and liposomes comprising DOPC.

2-4. (canceled)

5. A method of for protection against or prevention of RSV disease in a human subject of 70 years of age or older having a pre-existing medical condition selected from a chronic cardiovascular or pulmonary disease or an endocrine or metabolic condition, comprising the step of administration to the subject a composition comprising (i) a trimeric RSV F protein comprising the amino acid sequence of SEQ ID NO:2; and (ii) an adjuvant comprising QS21, cholesterol, 3D-MPL and liposomes comprising DOPC.

6-8. (canceled)

9. The method according to claim 5, wherein the pre-existing medical condition is selected from diabetes mellitus type 1 or type 2, chronic heart failure, advanced liver disease, advanced renal disease COPD, asthma or hypertension.

10. A method of protection against, or prevention of RSV disease in a human subject according to claim 1, wherein the RSV disease is caused by RSV A and/or RSV B in, and wherein the trimeric RSV F protein is derived from RSV A and the composition does not comprise an RSV F protein derived from RSV B.

11-13. (canceled)

14. The method according to claim 10, wherein each monomer of the trimeric RSV F protein comprises the amino acid sequence of SEQ ID NO:2

15. A method of protection against, or the prevention RSV acute respiratory infection (ARI), RSV lower respiratory tract disease (LRTD) or RSV severe lower respiratory tract disease (RSV severe LRTD) in a human subject of 70 years of age or older comprising the step of administering to the subject a composition comprising a trimeric RSV F protein wherein each monomer of the trimeric RSV F protein comprises the amino acid sequence of SEQ ID NO:2, and an adjuvant comprising QS21, cholesterol, 3D-MPL and liposomes comprising DOPC.
